SEQUENCE LISTING (18 sequences)

SEQ ID NO: 1 - protein, 10 aa, Artificial sequence
  Synthesised protein
SLFELGKMIL

SEQ ID NO: 2 - protein, 10 aa, Artificial sequence
  Synthesised protein
  MISC_FEATURE (10)..(10): Xaa = Leu-NH2
SLFELGKMIX

SEQ ID NO: 3 - protein, 11 aa, Artificial sequence
  Synthesised protein
SLFELGKMILQ

SEQ ID NO: 4 - protein, 11 aa, Artificial sequence
  Synthesised protein
  MISC_FEATURE (11)..(11): Xaa = Gln-NH2
SLFELGKMILX

SEQ ID NO: 5 - protein, 12 aa, Artificial sequence
  Synthesised protein
SLFELGKMILQE

SEQ ID NO: 6 - protein, 12 aa, Artificial sequence
  Synthesised protein
  MISC_FEATURE (12)..(12): Xaa = Glu-NH2
SLFELGKMILQX

SEQ ID NO: 7 - protein, 13 aa, Artificial sequence
  Synthesised protein
SLFELGKMILQET

SEQ ID NO: 8 - protein, 13 aa, Artificial sequence
  Synthesised protein
  MISC_FEATURE (13)..(13): Xaa = Thr-NH2
SLFELGKMILQEX

SEQ ID NO: 9 - protein, 20 aa, Artificial sequence
  Synthesised protein
SLFELGKMILQETGKNPAKS

SEQ ID NO: 10 - protein, 18 aa, Artificial sequence
  Synthesised protein
SLFELGKMILQETGKNPA

SEQ ID NO: 11 - protein, 16 aa, Artificial sequence
  Synthesised protein
SLFELGKMILQETGKN

SEQ ID NO: 12 - protein, 14 aa, Artificial sequence
  Synthesised protein
SLFELGKMILQETG

SEQ ID NO: 13 - protein, 8 aa, Artificial sequence
  Synthesised protein
SLFELGKM

SEQ ID NO: 14 - protein, 6 aa, Artificial sequence
  Synthesised protein
SLFELG

SEQ ID NO: 15 - protein, 4 aa, Artificial sequence
  Synthesised protein
SLFE

SEQ ID NO: 16 - protein, 10 aa, Artificial sequence
  Synthesised protein
KILGILFMSE

SEQ ID NO: 17 - protein, 9 aa, Artificial sequence
  Synthesised protein
  MISC_FEATURE (1)..(1): Xaa = (7-methoxycoumarin-4-yl)acetyl-Arg
  MISC_FEATURE (9)..(9): Xaa = Lys-(2,4-dinitrophenyl)
XPPGFSAFX

SEQ ID NO: 18 - protein, 7 aa, Artificial Sequence
  Synthesised protein
  MISC_FEATURE (1)..(1): Xaa = (7-methoxycomarin-4-yl)acetyl Pro
  MISC_FEATURE (4)..(4): Xaa = (L-norvaline) (3-(2,4-dinitrophenyl)
  MISC_FEATURE (5)..(5): Xaa = L-2,3-diaminopropionyl
  MISC_FEATURE (7)..(7): Xaa = Arg-NH2
XLAXXAX

(19) United States
(12) Patent Application Publication
(10) Pub. No.: US 2024/0002441 A1
(43) Pub. Date: Jan. 4, 2024

(54) PEPTIDES AND USES THEREOF IN MODULATION OF AMYLOID-BETA PROTEIN DEGRADING PROTEASES

(71) Applicants: THE UNIVERSITY OF QUEENSLAND, Brisbane, Queensland (AU); MONASH UNIVERSITY, Clayton, Victoria (AU)

(72) Inventors: Niwanthi Wickramasekara RAJAPAKSE, Taringa, Queensland (AU); Don Monath Sanjaya KURUPPU, Taringa, Queensland (AU); Alexander Ian SMITH, Hampton, Victoria (AU)

(21) Appl. No.: 18/254,782

(22) PCT Filed: Nov. 26, 2021
(86) PCT No.: PCT/AU2021/051415

§ 371 Date: May 26, 2023

(30) Foreign Application Priority Data
Nov. 26, 2020 (AU) 2020904375

(51) Int. Cl. C07K 7/08; A61P 25/28; A61K 45/06; A61P 3/10

(52) U.S. Cl. CPC C07K 7/08; A61P 25/28; A61K 45/06; A61P 3/10

(57) ABSTRACT

Described herein are peptides of formula (I) and pharmaceutical compositions and kits comprising the peptides. The use of the peptides in methods of treating or preventing diseases that are associated with modulation of Amyloid-beta protein-degrading proteases, such as neprilysin and angiotensin-converting enzyme 2, are also described.

### FIELD

The present invention relates to peptides. The present invention also relates to pharmaceutical compositions and kits comprising the peptides. The present invention further relates to the use of the peptides in methods of treating or preventing diseases that are associated with modulation of Amyloid-beta protein-degrading proteases, such as neprilysin and angiotensin-converting enzyme 2, are also described.

### BACKGROUND

Amyloid-beta protein-degrading proteases (AβDPs) are members of the M13 family of Zn²⁺-dependent proteases and include neprilysin (NEP), angiotensin-converting enzyme 1 and 2 (ACE1 and ACE2), insulin-degrading enzyme (IDE) and endothelin-converting enzyme 1 and 2 (ECE1, ECE2). AβDPs have been implicated in various physiological and pathophysiological roles. Therefore, there is interest in developing drugs which can modulate the activity of certain AβDPs for treating or preventing diseases.

One such AβDP is NEP, which has been implicated in Alzheimer's disease. Alzheimer's disease is a widespread neurological disorder that is characterised by a progressive decrease in cognitive function. The majority of Alzheimer's disease cases are ‘sporadic late-onset’ and affect patients 65 years of age and older. There is significant interest in developing therapies for treating Alzheimer's disease. However, the causes of Alzheimer's disease are poorly understood which has hampered the development of therapeutics. While some therapies have been developed, these therapies are for symptomatic treatment only and there are currently no therapies available which prevent or reverse the progression of Alzheimer's disease.

Abnormal accumulation of amyloid-beta protein (Aβ) in the brain is a hallmark of Alzheimer's disease. Recent research indicates that mechanisms of brain Aβ clearance may make a significant contribution to maintaining Aβ homeostasis, and failure of these mechanisms can drive Aβ accumulation as seen in ‘sporadic late-onset’ Alzheimer's disease. A mechanism for removal of brain Aβ which has recently been explored is breakdown by Aβ-degrading proteases (AβDPs), in particular NEP. Matrix metalloproteinases-2 and -9 have been shown to play a role in preventing Aβ accumulation in the brain.

NEP is a largely membrane bound protease and has been implicated as a catabolic regulator of Aβ homeostasis. Recent animal studies indicate the reduced NEP expression may be responsible at least in part for impaired clearance of Aβ observed in ‘sporadic late-onset’ Alzheimer's disease. In addition, increasing the expression of AβDPs, in particular NEP, can prevent Aβ build up and improve behaviour. These studies indicate that therapeutics which can increase the expression or activity of NEP could restore Aβ homeostasis and therefore prevent Aβ build up and disease onset. It may also be beneficial to increase the expression or activity of NEP selectively over certain related AβDPs, such as its closest homologue ECE1 which produces the potent vasoconstrictor endothelin-1, to avoid possible adverse effects. However, there are currently no drug therapies available that stimulate NEP activity.

Another AβDP of interest is ACE2. ACE2 is widely expressed in lungs, endothelial cells, kidney, heart and intestines. ACE2 breaks down Angiotensin II (Ang II) to produce Ang 1-7. Ang II is a potent vasoconstrictor with well documented fibrotic and inflammatory effects. In contrast, Ang 1-7 is a vasodilator and has anti-fibrotic and anti-inflammatory effects. Therefore, ACE2 is widely recognised as a negative regulator of the effects of Ang II and has been pursued as a potential target for treating diseases such as cardiovascular disease and renovascular disease.

There has also been interest in developing stimulators of ACE2 in the treatment or prevention of coronavirus infection, particularly in view of the emergence and global spread of severe acute respiratory syndrome coronavirus 2 (SARS-CoV-2). SARS-CoV-2 is highly infectious and the estimated mortality rate is 1-5%. Patients infected with SARS-CoV-2 display a range of symptoms including cough, fever, pneumonia and shortness of breath. The disease has also paralysed the global economy.

Coronaviruses such as SARS-CoV-2 are known to enter human cells by attaching to ACE2 present on the surface of the cells. For example, the spike protein of SARS-CoV-2 binds to human ACE2 (hACE2) present on the surface of cells which allows the virus to gain entry into the cells. Therefore, there is interest in developing drugs that can bind to ACE2 and prevent the coronavirus spike protein from binding to cells, which may thereby prevent entry of the coronavirus into cells. However, there are currently no known drugs that stimulate ACE2 activity.

Accordingly, there is a need for therapies that can be used for the treatment or prevention of diseases associated with AβDPs such as Alzheimer's disease, cardiovascular and renovascular disease, inflammation, fibrotic diseases and coronavirus infection.

### SUMMARY

The present invention is predicated at least in part on the discovery of peptides that can stimulate NEP activity and may be useful in the treatment or prevention of Alzheimer's disease. The present invention is also predicated at least in part on the discovery of peptides that can stimulate ACE2 activity and may be useful in the treatment or prevention of inflammation, fibrotic diseases, cardiovascular and/or renovascular disease, and/or may be useful in the treatment or prevention of a coronavirus infection.

In one aspect, there is provided a peptide of formula (I):

R₁-Xaa₁-Xaa₂-Xaa₃-Xaa₄-Xaa₅-Xaa₆-Xaa₇-Xaa₈-Xaa₋₉-Xaa₁₀-Xaa₁₁-Xaa₁₂-Xaa₁₃-R₂ (I)
or a pharmaceutically acceptable salt thereof,
wherein:
R₁ is —NH₂;
R₂ is —COR₃, wherein R₃ is selected from —OR₄ and —NHR₄, wherein R₄ is selected from H and C₁₋₃alkyl;

Xaa₁ is absent or is a polar uncharged α-amino acid;
Xaa₂ is leucine or alanine;
Xaa₃ is phenylalanine;
Xaa₄ is glutamic acid;
Xaa₅ is a hydrophobic α-amino acid or is a hydrophobic β-amino acid;
Xaa₆ is any α-amino acid;
Xaa₇ is lysine;
Xaa₈ is a hydrophobic α-amino acid;
Xaa₉ is a hydrophobic α-amino acid;
Xaa₁₀ is leucine;
Xaa₁₁ is absent or is a hydrophilic α-amino acid;
Xaa₁₂ is absent or is a negatively charged α-amino acid; and
Xaa₁₃ is absent or is selected from serine, threonine and cysteine.

In another aspect, there is provided a pharmaceutical composition comprising the peptide described herein or a pharmaceutically acceptable salt thereof and at least one pharmaceutically acceptable carrier.

In another aspect, there is provided a method for treating or preventing Alzheimer's disease comprising administering to a patient in need thereof the peptide described herein or a pharmaceutically acceptable salt thereof or the pharmaceutical composition described herein.

In another aspect, there is provided the use of the peptide described herein or a pharmaceutically acceptable salt thereof or the pharmaceutical composition described herein for treating or preventing Alzheimer's disease.

In another aspect, there is provided the use of the peptide described herein or a pharmaceutically acceptable salt thereof in the manufacture of a medicament for treating or preventing Alzheimer's disease.

In another aspect, there is provided the peptide described herein or a pharmaceutically acceptable salt thereof for use in treating or preventing Alzheimer's disease.

In another aspect, there is provided a method for treating or preventing inflammation, fibrosis, lung disease, hypertension, pulmonary hypertension, cardiovascular disease and/or renovascular disease comprising administering to a patient in need thereof the peptide described herein or a pharmaceutically acceptable salt thereof or the pharmaceutical composition described herein.

In another aspect, there is provided the use of the peptide described herein or a pharmaceutically acceptable salt thereof or the pharmaceutical composition described herein for treating or preventing inflammation, fibrosis, lung disease, hypertension, pulmonary hypertension, cardiovascular disease and/or renovascular disease.

In another aspect, there is provided the use of the peptide described herein or a pharmaceutically acceptable salt thereof in the manufacture of a medicament for treating or preventing inflammation, fibrosis, lung disease, hypertension, pulmonary hypertension, cardiovascular disease and/or renovascular disease.

In another aspect, there is provided the peptide described herein or a pharmaceutically acceptable salt thereof for use in treating or preventing inflammation, fibrosis, lung disease, hypertension, pulmonary hypertension, cardiovascular disease and/or renovascular disease.

In another aspect, there is provided a method for treating or preventing a coronavirus infection comprising administering to a patient in need thereof the peptide described herein or a pharmaceutically acceptable salt thereof or the pharmaceutical composition described herein.

In another aspect, there is provided the use of the peptide described herein or a pharmaceutically acceptable salt thereof or the pharmaceutical composition described herein for treating or preventing a coronavirus infection.

In another aspect, there is provided the use of the peptide described herein or a pharmaceutically acceptable salt thereof in the manufacture of a medicament for treating or preventing a coronavirus infection.

In another aspect, there is provided the peptide described herein or a pharmaceutically acceptable salt thereof for use in treating or preventing a coronavirus infection.

In another aspect, there is provided a kit comprising:
the peptide described herein or a pharmaceutically acceptable salt thereof or the pharmaceutical composition comprising a peptide described herein; and
an additional active agent useful in the treatment or prevention of Alzheimer's disease, inflammation, fibrosis, lung disease, hypertension, pulmonary hypertension, cardiovascular disease, renovascular disease and/or coronavirus infection.

### BRIEF DESCRIPTION OF THE DRAWINGS

FIG. 1 provides graphs illustrating the effect of SEQ ID NOS: 1, 5 and 9-15 on NEP and ECE1 activity. The vertical dotted lines indicate the activity of enzyme alone. The symbol (*) denotes significantly different compared to enzyme alone; n=4-6; one-way ANOVA; P<0.05.

FIG. 2 provides graphs illustrating the effect of increasing concentrations of 0.9-26 μM SEQ ID NO:2 (FIG. 2a), 9 μM SEQ ID NO:2 (FIG. 2b) and 2.6 μM SEQ ID NO:2 (FIG. 2c) on the activity of Aβ degrading enzymes NEP, ACE1, ACE2, IDE, ECE1, ECE2. Each data point represents a mean of 3-10 independent experiments and standard error of the mean. In FIG. 2b, the symbol (*) denotes significantly different from NEP+SEQ ID NO:2 (9 μM) and the symbol (#) denotes significantly different compared with respective enzyme alone. In FIG. 2c, the symbol (*) denotes significantly different compared with respective enzyme alone.

FIG. 3 provides graphs illustrating levels of Aβ1-40 (FIG. 3a), Aβ1-21 (FIG. 3b) and Aβ1-12 (FIG. 3c) detected by LCMS monitoring Aβ40 cleavage by NEP in the presence of SEQ ID NO:2 (squares), scrambled control (up triangles) or NEP alone (down triangles). Where indicated, peak area is expressed as % of initial. The symbol (#) denotes significantly different compared with NEP alone; P<0.05, one-way ANOVA, n=3-4. The symbol (*) denotes significantly different compared to NEP+scrambled control; P<0.05, unpaired t-test, n=3-4.

FIG. 4 provides graphs illustrating levels of Aβ1-12 (FIG. 4a), Aβ1-16 (FIG. 4b) and Aβ1-21 (FIG. 4c) detected by LCMS monitoring Aβ42 by NEP in the presence of SEQ ID NO:2 (squares), scrambled control (up triangles) or NEP alone (down triangles). The symbol (*) denotes significantly different compared with NEP alone; P<0.05, unpaired t-test, n=4. The symbol (*) denotes significantly different compared to NEP+scrambled control at t=24 h; P<0.05, one-way ANOVA, n=3-4.

FIG. 5 provides graphs illustrating Ang II-induced expression of IL-6 (FIG. 5a) and Collagen III (FIG. 5b) in the presence of SEQ ID NO:2, rhACE2, or a combination of SEQ ID NO:2, rhACE2 and ACE2 inhibitor MLN4760. The symbol (#) denotes significantly different to Ang II and the symbol (*) denotes significantly different compared to vehicle; P≤0.5, unpaired t-test, n=3.

FIG. 6 provides graphs illustrating viral titre (FIG. 6a) and percentage viral RNA (FIG. 6b) in Vero cells pre-treated with SEQ ID NO:2 at 200 ng/μL or 400 ng/μL or PBS, followed by SARS-CoV-2 infection. Each data point represents data derived from individual wells and is pooled from a minimum of two independent experiments. In FIG. 6a, the symbol (*) denotes significantly different compared to PBS; P<0.05 by one-way ANOVA. In FIG. 6b, the symbol (*) denotes significantly different compared to PBS; P<0.001 by one sample t-test.

FIG. 7 provides graphs illustrating viral titre (FIG. 7a), percentage viral RNA (FIG. 7b) and relative IL-6 expression (FIG. 7c) in Vero cells infected with SARS-CoV-2, followed by treatment with SEQ ID NO:2 at 200 ng/μL or 400 ng/μL or PBS. Each data point represents data derived from individual wells and is pooled from a minimum of two independent experiments. In FIG. 7a, the symbol (*) denotes significantly different compared to PBS; P=0.0151 by Kruskal-Wallis test. In FIG. 7b, the symbol (*) denotes significantly different compared to PBS; P=0.0322 (200 ng/μL SEQ ID NO:2) and P=0.0051 (400 ng/μL SEQ ID NO:2) by one sample t-test. In FIG. 7c, the expression of IL-6 is shown relative to PBS-treated cells and normalised to GAPDH; and the symbols (*) and (**) denote significantly different compared to PBS; P=(200 ng/μL SEQ ID NO:2) and P=0.0022 (400 ng/μL SEQ ID NO:2), respectively, by Kruskal-Wallis test.

FIG. 8 provides graphs illustrating the change in average body weight over time (8a), change in average blood glucose over time (8b), kidney IL-6 expression (8c), kidney collagen I expression (8d) and kidney collagen III expression (8e) in an STZ mouse model, where the mice were treated with SEQ ID NO:2 or vehicle.

FIG. 9 provides graphs illustrating the effects of SEQ ID NO. 2 (referred to in FIG. as 2A) (1 mg/kg/day; 10% DMSO/PBS; s.c. osmotic mini-pump) in ND (non-diabetic) mice and STZ mice on (a) body weight, (b) blood glucose levels over time, (c) urine volume over time, (d) kidney:body weight ratio, (e) changes in 24 h urinary albumin levels, (f) percentage of fibrosis in the renal cortical region, and (g) IL-6 protein expression in the kidney of STZ+SEQ ID NO. 2 (2A) and STZ+vehicle mice after 4 weeks of treatment (n=3-7). Unpaired Student's t-test. Data are mean±SEM.

FIG. 10 provides graphs illustrating the effects of treatment of wild type (WT) and 5×FAD transgenic (Tg) female mice treated for one month with vehicle (10% DMSO/PBS) or SEQ ID NO. 2 (Referred to in Figure as 2A) (1 mg/kg). (a) Body weights of the mice for duration of 1.5-month experiment, n=6-9/group. (b) % time the mice spent in centre zone during open field/activity monitor behavioural test, pre-treatment compared with post-treatment (paired t-test, n=6-9/group). (c) Frequency of entry of the mice to the novel arm in y-maze behavioural test for spatial reference memory, as % of entries to all other arms, pre-treatment compared with post-treatment (paired t-test, n=6-9/group). (d) Number of shocks received by each group on day 5 of testing for the active place avoidance behavioural test, pre- and post-treatment, *significantly different vs pre-treatment (P<0.03; paired t-test). (e) Number of shocks received by each group post-treatment in the active place avoidance behavioural test. (f) Comparison of 5-day post-treatment performance of the transgenic mice treated with vehicle or SEQ ID NO. 2 (2A) in active place avoidance behavioural testing, measured using (from left to right) (i) number of shocks received, (ii) time to first entry to shock zone (s), (iii) maximum time avoidance of shock zone (s), and (iv) number of entrances to shock zone. Protein levels of inflammatory markers (g) Interleukin-6 (IL-6) and (h) tumour necrosis factor-α (TNF-α) expression in cortex and hippocampal tissue of female mice measured by western blotting, unpaired t-test, n=3-7. (i) Protein levels of synaptic marker synaptotagmin in cortex and hippocampal tissue of female mice measured by western blotting, unpaired t-test, n=3-7.

### DETAILED DESCRIPTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

As used herein, the term “about” refers to a quantity, value, dimension, size, or amount that varies by as much as 30%, 25%, 20%, 15% or 10% to a reference quantity, value, dimension, size, or amount.

As used herein, unless the context requires otherwise, the term “comprise”, and variations such as “comprises” and “comprising”, will be understood to imply the inclusion of a stated integer or step or group of integers or steps but not the exclusion of any other integer or step or group of integers or steps.

The term “amino acid” as used herein refers to an α-amino acid or a β-amino acid and may be a L- or D-isomer. The amino acid may have a naturally occurring side chain (see Table 1) or a non-proteinogenic side chain. The amino acid may also be further substituted in the α-position or the β-position with a group selected from —C₁₋₆alkyl, —(CH₂)ₙCOR^{a}, —(CH₂)ₙR^{b} and —PO₃H, where n is an integer selected from 1 to 8, R^{a} is —OH, —NH₂, —NHC₁₋₃alkyl, —OC₁₋₃alkyl or —C₁₋₃alkyl and R_{b} is —OH, —SH, —SC₁₋₃alkyl, —OC₁₋₃alkyl, —NH₂, —NHC₁₋₃alkyl or —NHC(C═NH)NH₂ and where each alkyl group may be substituted with one or more groups selected from —OH, —NH₂, —NHC₁₋₃alkyl, —OC₁₋₃alkyl, —SH, —SC₁₋₃alkyl, —CO₂H, —CO₂C₁₋₃alkyl, —CONH₂ and —CONHC₁₋₃alkyl.

Amino acid structure and single and three letter abbreviations used throughout the specification are defined in Table 1, which lists the twenty proteinogenic naturally occurring amino acids which occur in proteins as L-isomers.

TABLE 1

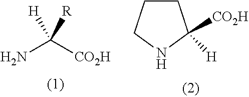

| | Three-letter | One-letter | Structure of side chain |
|---|---|---|---|
| Amino Acid | Abbreviation | symbol | (R) |
| Alanine | Ala | A | —CH₃ |
| Arginine | Arg | R | —(CH₂)₃NHC(═N)NH₂ |
| Asparagine | Asn | N | —CH₂CONH₂ |
| Aspartic acid | Asp | D | —CH₂CO₂H |
| Cysteine | Cys | C | —CH₂SH |
| Glutamine | Gln | Q | —(CH₂)₂CONH₂ |
| Glutamic acid | Glu | E | —(CH₂)₂CO₂H |
| Glycine | Gly | G | —H |
| Histidine | His | H | —CH₂(4-imidazolyl) |
| Isoleucine | Ile | I | —CH(CH₃)CH₂CH₃ |
| Leucine | Leu | L | —CH₂CH(CH₃)₂ |
| Lysine | Lys | K | —(CH₂)₄NH₂ |
| Methionine | Met | M | —(CH₂)₂SCH₃ |
| Phenylalanine | Phe | F | —CH₂Ph |
| Proline | Pro | P | see formula (2) above for |
| | | | structure of amino acid |
| Serine | Ser | S | —CH₂OH |
| Threonine | Thr | T | —CH(CH₃)OH |
| Tryptophan | Trp | W | —CH₂(3-indolyl) |
| Tyrosine | Tyr | Y | —CH₂(4-hydroxyphenyl) |
| Valine | Val | V | —CH(CH₃)₂ |

The term “non proteinogenic amino acid” as used herein refers to an amino acid having a side chain that does not occur in the naturally occurring L-α-amino acids recited in Table 1. Examples of non-proteinogenic amino acids and derivatives include, but are not limited to, norleucine, 4-aminobutyric acid, 4-amino-3-hydroxy-5-phenylpentanoic acid, 6-aminohexanoic acid, t-butylglycine, norvaline, phenylglycine, ornithine, citrulline, sarcosine, 4-amino-3-hydroxy-6-methylheptanoic acid, 2-thienyl alanine and/or D-isomers of natural amino acids.

The term “α-amino acid” as used herein refers to an amino acid that has a single carbon atom (the α-carbon atom) separating a carboxyl terminus (C-terminus) and an amino terminus (N-terminus). An α-amino acid includes naturally occurring and non-naturally occurring L-amino acids and their D-isomers and derivatives thereof such as salts or derivatives where functional groups are protected by suitable protecting groups.

The term “β-amino acid” as used herein refers to an amino acid that differs from an α-amino acid in that there are two (2) carbon atoms separating the carboxyl terminus and the amino terminus. As such, β-amino acids with a specific side chain can exist as the R or S enantiomers at either of the α (C2) carbon or the β (C3) carbon, resulting in a total of 4 possible isomers for any given side chain. The side chains may be the same as those of naturally occurring α-amino acids (see Table 1 above) or may be the side chains of non-naturally occurring amino acids.

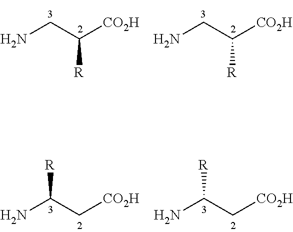

Suitable derivatives of β-amino acids include salts and may have functional groups protected by suitable protecting groups.

The term “hydrophobic amino acid” as used herein refers to an amino acid having a side chain which is non-polar. Examples include, but are not limited to, glycine, alanine, valine, leucine, isoleucine, proline, methionine, phenylalanine, tryptophan, aminoisobutyric acid, cyclohexylalanine, cyclopentylalanine, norleucine, norvaline, tert-butylglycine and ethylglycine, especially alanine, valine, leucine, isoleucine, proline, methionine, phenylalanine, tryptophan and aminoisobutyric acid. The amino acid may be an α-amino acid or a β-amino acid.

The term “hydrophilic amino acid” as used herein refers to an amino acid having a side chain which is polar or charged. Examples include, but are not limited to, serine, threonine, cysteine, tyrosine, asparagine, glutamine, aspartic acid, glutamic acid, lysine, arginine, histidine and ornithine. The amino acid may be an α-amino acid or a β-amino acid.

The term “polar uncharged amino acid” refers to an amino acid having a side chain that has a dipole moment. Examples include, but are not limited to, serine, threonine, cysteine, tyrosine, asparagine and glutamine. The amino acid may be an α-amino acid or a β-amino acid.

The term “positively charged amino acid” refers to an amino acid having a side chain capable of bearing a positive charge. Examples include, but are not limited to, lysine, arginine, histidine and ornithine. The amino acid may be an α-amino acid or a β-amino acid.

The term “negatively charged amino acid side chain” refers to an amino acid having a side chain capable of bearing a negative charge. Examples include, but are not limited to, aspartic acid and glutamic acid. The amino acid may be an α-amino acid or a β-amino acid.

Those skilled in the art will appreciate that a peptide represents a series of two or more amino acids linked through a covalent bond formed between the carboxyl group of one amino acid and the amino group of another amino acid (i.e. the so-called peptide bond).

The term “alkyl” as used herein refers to straight chain or branched hydrocarbon groups. Where appropriate, the alkyl group may have a specified number of carbon atoms, for example, C₁₋₃alkyl which includes alkyl groups having 1, 2 or 3 carbon atoms in a linear or branched arrangement. Examples of suitable alkyl groups include methyl, ethyl, n-propyl and i-propyl.

Additional definitions are provided in the description below.

### 2. Peptides

There is provided a peptide of formula (I):

R₁-Xaa₁-Xaa₂-Xaa₃-Xaa₄-Xaa₅-Xaa₆-Xaa₇-Xaa₈-Xaa₋₉-Xaa₁₀-Xaa₁₁-Xaa₁₂-Xaa₁₃-R₂ (I)
wherein:
R₁ is —NH₂;
R₂ is —COR₃, wherein R₃ is selected from —OR₄ and —NHR₄, wherein R₄ is selected from H and C₁₋₃alkyl;
Xaa₁ is absent or is a polar uncharged α-amino acid;
Xaa₂ is leucine or alanine;
Xaa₃ is phenylalanine;
Xaa₄ is glutamic acid;
Xaa₅ is a hydrophobic α-amino acid or is a hydrophobic β-amino acid;
Xaa₆ is any α-amino acid;
Xaa₇ is lysine;
Xaa₈ is a hydrophobic α-amino acid;
Xaa₉ is a hydrophobic α-amino acid;
Xaa₁₀ is leucine;
Xaa₁₁ is absent or is a hydrophilic α-amino acid;
Xaa₁₂ is absent or is a negatively charged α-amino acid; and
Xaa₁₃ is absent or is selected from serine, threonine and cysteine.

In some embodiments, the C-terminus (—R₂) is a free acid (—COOH). In some embodiments, the C-terminus may be a derivative or analogue of a free acid group, for example an ester (—COOC₁₋₃alkyl) or a primary or secondary amide (—CONHR₄ wherein R₄ is selected from H and C₁₋₃alkyl). Advantageously, having a C-terminus that is a derivative or analogue of a free acid group may improve the biological stability of the peptide compared to the free acid.

The peptide may be susceptible to proteolytic cleavage at the peptide bond between Xaa₄ and Xaa₅. Accordingly, in some embodiments, Xaa₅ is a non-proteinogenic amino acid capable of preventing or reducing cleavage of the peptide by proteases, for example α-aminoisobutyric acid or a β-amino acid.

In some embodiments, the peptides may selectively stimulate the activity of one or more Aβ-degrading proteases. By the term “selective”, it is meant that the peptide binds to and/or stimulates the activity of one or more Aβ-degrading proteases to a greater extent than binding and stimulation of one or more other Aβ-degrading proteases. In some instances, selective refers to binding and/or activation of the one or more Aβ-degrading proteases with little or no binding and/or minimal or no stimulating effect at the other Aβ-degrading proteases. In particular embodiments, the peptides have stimulatory activity at one or more of NEP, ACE1 and ACE2, especially NEP and ACE2, and minimal or no activity with one or more other Aβ-degrading proteases, selected from endothelin-converting enzyme 1 and 2 (ECE1, ECE2) and insulin-degrading enzyme (IDE), especially ECE1. In particular embodiments, the peptides selectively stimulate NEP while having no or minimal effect on structurally related enzyme ECE1.

In some embodiments of formula (I), one or more of the following applies:
R₂ is —COOH or —CONH₂;
Xaa₁ is a polar uncharged α-amino acid, especially serine or threonine, more especially serine;
Xaa₂ is leucine;
Xaa₅ is selected from alanine, isoleucine, leucine, valine, α-aminoisobutyric acid, β-homoalanine, β-homoleucine, β-homoisoleucine and β-leucine, especially leucine or isoleucine, more especially leucine;
Xaa₆ is a hydrophobic α-amino acid, especially glycine, alanine, valine, leucine or isoleucine, more especially glycine or alanine, most especially glycine;
Xaa₈ is selected from isoleucine, leucine, methionine and valine, especially methionine;
Xaa₉ is selected from alanine, isoleucine, leucine and valine, especially isoleucine or leucine, more especially isoleucine;
Xaa₁₁ is absent;
or Xaa₁₁ is a hydrophilic α-amino acid, especially a polar uncharged α-amino acid, more especially selected from serine, threonine, asparagine and glutamine, even more especially glutamine;
Xaa₁₂ is absent;
or Xaa₁₂ is a negatively charged α-amino acid, especially glutamic acid or aspartic acid, more especially glutamic acid; and
Xaa₁₃ is absent;
or Xaa₁₃ is selected from serine, threonine and cysteine, especially threonine.

In particular embodiments, the peptide of formula (I) is selected from:

SEQ ID NO: 1
H₂N-SLFELGKMIL-OH
SEQ ID NO: 2
H₂N-SLFELGKMIL-NH₂
SEQ ID NO: 3
H₂N-SLFELGKMILQ-OH
SEQ ID NO: 4
H₂N-SLFELGKMILQ-NH₂
SEQ ID NO: 5
H₂N-SLFELGKMILQE-OH
SEQ ID NO: 6
H₂N-SLFELGKMILQE-NH₂
SEQ ID NO: 7
H₂N-SLFELGKMILQET-OH
SEQ ID NO: 8
H₂N-SLFELGKMILQET-NH₂.

The peptides may be in the form of pharmaceutically acceptable salts. It will be appreciated however that non-pharmaceutically acceptable salts also fall within the scope since these may be useful as intermediates in the preparation of pharmaceutically acceptable salts or may be useful during storage or transport. Suitable pharmaceutically acceptable salts include, but are not limited to, salts of pharmaceutically acceptable inorganic acids such as hydrochloric, sulfuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benezenesulphonic, salicylic sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, ammonium and alkylammonium.

Basic nitrogen-containing groups may be quaternised with such agents as lower alkyl halide, such as methyl, ethyl, propyl, and butyl chlorides, bromides and iodides; dialkyl sulfates like dimethyl and diethyl sulfate; and others.

The peptides may also be modified to enhance pharmacokinetic and/or pharmacodynamic properties. For example, the peptides may be modified by conjugation with an entity that modifies liberation, absorption, distribution, metabolism and/or excretion of the peptide. One example of a pharmacokinetic modifying moiety is polyethylene glycol (PEG). Pegylation of the peptide may increase residence time in the circulatory system by reducing renal clearance of the peptide, may increase solubility and/or may reduce immunogenicity of the peptide.

The peptides may be prepared by known methods, including solid-phase and solution-phase peptide synthesis using Fmoc or Boc protected amino acid residues.

As shown in the Examples, the peptides are capable of stimulating NEP activity. Accordingly, the peptides may be useful in the treatment or prevention of Alzheimer's disease. As also shown in the Examples, the peptides are capable of stimulating ACE2 activity. Accordingly, the peptides may also be useful in the treatment or prevention of inflammation, fibrosis, cardiovascular diseases and/or renovascular diseases. The peptides may further be useful in the treatment or prevention of a coronavirus infection, including symptoms associated with coronavirus infection. Advantageously, as shown in the Examples, the peptides of the present invention may selectively stimulate the activity of NEP and ACE2 while having no or minimal stimulating effect on the activity of structurally related enzyme ECE1.

### 3. Pharmaceutical Compositions and Kits

Also provided are pharmaceutical compositions comprising the peptide described herein and at least one pharmaceutically acceptable carrier.

The term “pharmaceutically acceptable carrier” refers to a solid or liquid filler, diluent, excipient, solvent or encapsulating substance that may be safely used in topical or systemic administration. The carrier(s) must be “acceptable” in the sense of being compatible with the other ingredients of the composition and not deleterious to the recipient thereof.

The pharmaceutical composition may be suitably formulated for administration by a particular route. Suitable routes of administration include oral, transmucosal, transdermal, and parenteral administration. In some embodiments, the composition is formulated for oral administration, topical administration such as buccal or sublingual administration, nasal administration, transdermal administration, or parenteral administration such as subcutaneous, intramuscular or intravenous administration. In preferred embodiments, the composition is formulated for oral administration, nasal administration, topical administration, especially sublingual administration, or parenteral administration, especially subcutaneous administration or intravenous administration, more especially intravenous administration.

Pharmaceutical formulations include those suitable for oral, nasal, topical (including buccal and sub-lingual) or parenteral (including intramuscular, sub-cutaneous and intravenous) administration or in a form suitable for administration by inhalation or insufflation. The peptides, together with a conventional adjuvant, carrier, excipient, or diluent, may thus be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use; or in the form of sterile injectable solutions for parenteral (including subcutaneous) use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed. The peptides can be administered in a wide variety of oral and parenteral dosage forms. It will be obvious to those skilled in the art that the following dosage forms may comprise, as the active component, either a peptide or a pharmaceutically acceptable salt or derivative of the peptide of the invention.

For preparing pharmaceutical compositions from the peptides, pharmaceutically acceptable carriers can be either solid or liquid. Solid form preparations include powders, tablets, pills, capsules, cachets, and dispersible granules. A solid carrier can be one or more substances which may also act as diluents, flavouring agents, solubilizers, lubricants, suspending agents, binders, preservatives, tablet disintegrating agents, or an encapsulating material.

In powders, the carrier is a finely divided solid which is in a mixture with the finely divided active component.

In tablets, the active component is mixed with the carrier having the necessary binding capacity in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

Liquid form preparations include solutions, suspensions, and emulsions. For example, parenteral injection liquid preparations can be formulated as solutions.

The peptides may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavours, stabilizing and thickening agents, as desired.

Aqueous suspensions suitable for oral use can be made by dispersing the finely divided active component in water with viscous material.

Also included are solid form preparations which are intended to be converted, shortly before use, to liquid form preparations for oral administration. Such liquid forms include solutions, suspensions, and emulsions. These preparations may contain, in addition to the active component, colorants, flavours, stabilizers, buffers, artificial and natural sweeteners, dispersants, thickeners, solubilizing agents, and the like.

For topical administration to the epidermis, the peptides may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or colouring agents.

Formulations suitable for topical administration in the mouth include lozenges comprising active agent in a flavoured base; pastilles comprising the active ingredient in an inert base; and mouthwashes comprising the active ingredient in a suitable liquid carrier.

Solutions or suspensions are applied directly to the nasal cavity by conventional means, for example with a dropper, pipette or spray. The formulations may be provided in single or multidose form. In the latter case of a dropper or pipette, this may be achieved by the patient administering an appropriate, predetermined volume of the solution or suspension. In the case of a spray, this may be achieved for example by means of a metering atomizing spray pump. To improve nasal delivery and retention, the peptides according to the invention may be encapsulated with cyclodextrins, or formulated with their agents expected to enhance delivery and retention in the nasal mucosa.

Administration to the respiratory tract may also be achieved by means of an aerosol formulation in which the active ingredient is provided in a pressurised pack with a suitable propellant such as a chlorofluorocarbon (CFC) for example, dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively, the active ingredient may be provided in the form of a dry powder, for example a powder mix of the active compound in a suitable powder base.

Conveniently, the powder carrier will form a gel in the nasal cavity. The powder composition may be presented in unit dose form for example in capsules or cartridges of, e.g., gelatin, or blister packs from which the powder may be administered by means of an inhaler.

In formulations intended for administration to the respiratory tract, including intranasal formulations, the active compound will generally have a small particle size for example of the order of 1 to 10 microns or less. Such a particle size may be obtained by means known in the art, for example by micronisation.

When desired, formulations adapted to give sustained release of the active ingredient may be employed.

The pharmaceutical preparations can be in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The pharmaceutical composition may further comprise an additional active agent useful in the treatment of Alzheimer's disease. Suitable active agents useful in the treatment of Alzheimer's disease include agents capable of facilitating the blood-brain barrier (BBB) permeability of peptides, including agents such as L-arginine or L-glutamine. Advantageously, administering the peptide in combination with L-arginine or L-glutamine may improve the BBB permeability of the peptide, which may improve the therapeutic effect of the peptide. Other useful agents include Aβ antioxidants such as branched-chain amino acids, L-glutamine, lipoic acid, urate, vitamin E, vitamin C, retinol and β-carotene, Other suitable agents include agents capable of decreasing the production and/or reducing the buildup of Aβ and/or tau protein.

Also provided are kits comprising the peptide described herein or the pharmaceutical composition described herein, and an additional active agent useful in the treatment of Alzheimer's disease. Suitable active agents useful in the treatment of Alzheimer's disease include those described herein. In some embodiments, the additional active agent is L-arginine or L-glutamine. In some embodiments, the additional active agent is an antioxidant.

### 4. Methods of Use

The peptides of formula (I), as stimulators of NEP, may be useful in the treatment of Alzheimer's disease. Accordingly, the present invention provides a method for treating or preventing Alzheimer's disease comprising administering to a patient in need thereof the peptide described herein or the pharmaceutical composition described herein. Also provided is the use of the peptide described herein for treating or preventing Alzheimer's disease. Also provided is the use of the peptide described herein in the manufacture of a medicament for treating or preventing Alzheimer's disease. Still further provided is the peptide described herein for use in treating or preventing Alzheimer's disease.

The term “treating Alzheimer's disease” in this context refers to an improvement in symptoms associated with Alzheimer's disease, where the improvement may be characterised qualitatively or quantitatively by assessments known in the art. The term “preventing Alzheimer's disease” in this context does not mean that the subject never suffers Alzheimer's disease but instead, the therapy may delay the onset of Alzheimer's disease. The subject may have a family history of Alzheimer's disease.

The methods and uses for treating or preventing Alzheimer's disease may further comprise administering an additional active agent useful in the treatment or prevention of Alzheimer's disease in combination with the peptide of formula (I). Suitable active agents useful in the treatment or prevention of Alzheimer's disease include those described herein. In some embodiments, the additional active agent is L-arginine. In some embodiments, the additional active agent is an antioxidant.

The additional active agent and the peptide may be administered together in a single composition or in separate compositions. Accordingly, in some embodiments, the additional active agent and the peptide of formula (I) are administered in a single composition, such as the pharmaceutical compositions described herein. In other embodiments, the additional active agent and the peptide of formula (I) are administered simultaneously or sequentially in separate compositions. Advantageously, administering the peptide of formula (I) in combination with L-arginine may improve the BBB permeability of the peptide, which may improve the therapeutic effect of the peptide. The peptide of formula (I) may be administered at different times and different frequencies, but in combination they exert biological effects at the same time or at overlapping times.

The peptides of formula (I), as stimulators of ACE2, may also be useful in the treatment or prevention of fibrosis, inflammation, lung disease, hypertension, pulmonary hypertension, cardiovascular disease and/or renovascular disease. Accordingly, there is provided a method for treating or preventing fibrosis, inflammation, lung disease, hypertension, pulmonary hypertension, cardiovascular disease and/or renovascular disease comprising administering to a patient in need thereof the peptide described herein or the pharmaceutical composition described herein. Also provided is the use of the peptide described herein for treating or preventing fibrosis, inflammation, lung disease, hypertension, pulmonary hypertension, cardiovascular disease and/or renovascular disease. Further provided is the use of the peptide described herein in the manufacture of a medicament for treating or preventing fibrosis, inflammation, lung disease, hypertension, pulmonary hypertension, cardiovascular disease and/or renovascular disease. Still further provided is the peptide described herein for use in treating or preventing fibrosis, inflammation, lung disease, hypertension, pulmonary hypertension, cardiovascular disease and/or renovascular disease.

The fibrosis may be fibrosis in any organ, for example, heart, kidney, lung, liver or skin. Similarly, inflammation may occur in any organ or tissue, for example in the heart, kidney, lung, liver and pancreas and may be chronic or acute. The inflammation may be associated with chronic disease such as metabolic syndrome, heart disease, kidney disease or with infections such as viral infections. In some embodiments, the inflammation is an inflammatory lung disease, for example, asthma, chronic obstructive pulmonary disease (COPD), sarcoidosis and pneumonia.

In addition to inflammatory lung disease, other lung diseases may be treated by the peptides, for example, bronchopulmonary dysplasia and pulmonary hypertension.

There is also significant data available that indicates that activating ACE2 can reduce blood pressure (hypertension), including pulmonary hypertension.

The term “cardiovascular disease” refers to a disease of the heart or blood vessels. Examples of cardiovascular disease include, but are not limited to, coronary artery disease, stroke and heart failure. The term “renovascular disease” refers to a disease of the arteries of the kidneys. Examples of cardiovascular disease and renovascular disease include diabetes related heart and kidney disease, hypertension and related kidney disease, chronic and acute kidney diseases, heart and kidney fibrosis, and heart and kidney inflammation. The term “treating cardiovascular disease and/or renovascular disease” in this context refers to an improvement in symptoms associated with cardiovascular disease and/or renovascular disease, where the improvement may be characterised qualitatively or quantitatively by assessments known in the art. The term “preventing cardiovascular disease and/or renovascular disease” in this context does not mean that the subject never suffers cardiovascular disease and/or renovascular disease but instead, the therapy may delay the onset of cardiovascular disease and/or renovascular disease.

The methods and uses may further comprise administering an additional active agent useful in the treatment or prevention of fibrosis, inflammation, lung disease, lung disease, hypertension, pulmonary hypertension, cardiovascular disease or renovasular disease in combination with the peptide of formula (I). Suitable active agents useful in the treatment or prevention of fibrosis, inflammation, cardiovascular disease or renovasular disease include those anti-fibrotic agents, anti-inflammatory agents such as non-steroidal anti-inflammatory agents, bronchodilators, Angiotensin II receptor blockers, angiotensin converting enzyme-1 inhibitors, thiazide diuretics and calcium channel blockers.

The additional active agent and the peptide may be administered together in a single composition or in separate compositions. Accordingly, in some embodiments, the additional active agent and the peptide of formula (I) are administered in a single composition, such as the pharmaceutical compositions described herein. In other embodiments, the additional active agent and the peptide of formula (I) are administered simultaneously or sequentially in separate compositions. The peptide of formula (I) may be administered at different times and different frequencies, but in combination they exert biological effects at the same time or at overlapping times.

The peptides of formula (I), as stimulators of ACE2, may further be useful in the treatment or prevention of a coronavirus infection. Accordingly, there is provided a method for treating or preventing coronavirus infection comprising administering to a patient in need thereof the peptide described herein or the pharmaceutical composition described herein. Also provided is the use of the peptide described herein for treating or preventing a coronavirus infection. Further provided is the use of the peptide described herein in the manufacture of a medicament for treating or preventing a coronavirus infection. Still further provided is the peptide described herein for use in treating or preventing a coronavirus infection.

In these embodiments, the coronavirus may be any coronavirus capable of entering cells (e.g. human cells) by binding ACE2 (e.g. human ACE2) via the spike protein of the coronavirus. Examples of such coronaviruses include human coronavirus NL63 (HCoV-NL63), severe acute respiratory syndrome coronavirus (SARS-CoV) and severe acute respiratory syndrome coronavirus 2 (SARS-CoV-2). In some embodiments, the coronavirus is SARS-CoV-2.

The term “treating a coronavirus infection” in this context includes an improvement in symptoms associated with the coronavirus infection, such as inflammation or fibrosis associated with the coronavirus infection, where the improvement may be characterised qualitatively or quantitatively by assessments known in the art. As shown in the Examples, the term “preventing a coronavirus infection” in this context does not mean that the subject is never infected by a coronavirus but instead, the therapy may delay coronavirus infection.

The methods and uses of treating a coronavirus infection may further comprise administering an additional active agent useful in the treatment or prevention of coronavirus in combination with the peptide of formula (I). Suitable active agents useful in the treatment or prevention of coronavirus include antiviral agents such as remdesivir or molnupiravir and/or anti-inflammatory agents such as dexamethasone.

The additional active agent and the peptide may be administered together in a single composition or in separate compositions. Accordingly, in some embodiments, the additional active agent and the peptide of formula (I) are administered in a single composition, such as the pharmaceutical compositions described herein. In other embodiments, the additional active agent and the peptide of formula (I) are administered simultaneously or sequentially in separate compositions. The peptide of formula (I) may be administered at different times and different frequencies, but in combination they exert biological effects at the same time or at overlapping times.

### EXAMPLES

The present invention will now be described with reference to the following non-limiting Examples.

The following materials were used in the Examples. Recombinant human (rh) NEP (Cat #1182-ZNC-010), ECE1 (Cat #1784-ZN-010), ECE2 (Cat #1645-ZN-010), and ACE2 (Cat #933-ZN-010) were purchased from R & D systems. MMP2 (Cat #SRP6270-10UG), ACE1 (Cat #SRP6270-10UG) and NEP inhibitor thiorphan (Cat #T6031) was purchased from Sigma Aldrich. Unless otherwise stated, all synthetic peptides were synthesised by Genic Bio Ltd (Shanghai, China). Synthetic amyloid-beta peptide 1-40 (Aβ40) (Cat #4014442) and amyloid-beta peptide 1-40 (Aβ42) (Cat #4014447) were purchased from Bachem.

### Example 1—Synthesis of Peptides

The following peptides were synthesised by Genic Bio Ltd (Shanghai, China) under instruction using standard Fmoc solid phase peptide synthesis procedures.

SEQ ID NO: 1
H₂N-SLFELGKMIL-OH
SEQ ID NO: 2
H₂N-SLFELGKMIL-NH₂
SEQ ID NO: 5
H₂N-SLFELGKMILQE-OH
SEQ ID NO: 9
H₂N-SLFELGKMILQETGKNPAKS-OH
SEQ ID NO: 10
H₂N-SLFELGKMILQETGKNPA-OH
SEQ ID NO: 11
H₂N-SLFELGKMILQETGKN-OH
SEQ ID NO: 12
H₂N-SLFELGKMILQETG-OH
SEQ ID NO: 13
H₂N-SLFELGKM-OH
SEQ ID NO: 14
H₂N-SLFELG-OH
SEQ ID NO: 15
H₂N-SLFE-OH
SEQ ID NO: 16
H₂N-KILGILFMSE-OH

SEQ ID NOS: 9-15 are used in the examples below as comparative peptides. SEQ ID NO:16 is a scrambled analogue of SEQ ID NO:1 and is used in the examples below as a negative control (“scrambled control”).

### Example 2—Method of Performing Plate Reader-Based Enzyme Assays

Plate reader-based enzyme assays were performed using a quenched fluorescent substrate (QFS) in a 96-well format using a SpectraMAX M4 Microplate reader (Molecular Devices). The increase in fluorescence following addition of QFS (40 μM) was taken as evidence of enzyme activity. Fluorescence was monitored over 2 h at 37° C. using κ_{Ex}=320 nm and λ_{Em}=405 nm. Assays were conducted in black 96-well plates. Specific enzyme activity was expressed as a rate of substrate cleavage calculated using a standard curve of 7-methoxy-coumarin-4-acetic acid. The specific QFS and buffers used with each enzyme are indicated in Table 2.

TABLE 2
Buffer compositions and respective quenched fluorescent substrate used for
each Aβ degrading enzyme.

| Enzyme | Buffer | Quenched fluorescence substrate |
|---|---|---|
| rhNEP | 50 mM TrisHC1, 150 mM NaCl; pH 6.3 | (7-methoxycoumarin-4-yl)acetyl-Arg- |
| rhIDE | 50 mM TrisHC1, 1 M NaCl; pH 7.5 | Pro-Pro-Gly-Phe-Ser-Ala-Phe-Lys-(2,4- |
| rhECE1 | 50 mM TrisHC1, 150 mM NaCl; pH 6.3 | dinitrophenyl)-OH (SEQ ID NO. 17) |
| rhECE2 | 100 mM MES, 250 mM NaCl; pH 5.75 | |
| rhACE1 | 50 mM HEPES, 300 mM NaCl; pH 8.3 | |
| rhACE2 | 100 mM Tris-HC1, 1 M NaCl; pH 6.5 | (7-methoxycomarin-4-yl)acetyl-Ala-Pro- |
| | | Lys-(2,4-dinitrophenyl)-OH |
| rhMMP2 | 50 mM Tris-HC1, 10 mM CaCl₂, 150 | (7-methoxycomarin-4-yl)acetyl-Pro-Leu- |
| | mM NaC1, 0.05% (w/v) Brij-35; pH 7.5 | Ala-(L-norvaline)-(3-(2,4- |
| | | dinitrophenyl)-L-2,3-diaminopropionyl- |
| | | Ala-Arg-NH₂ (SEQ ID NO. 18) |

In all plate reader-based enzyme assays using QFS, the reaction rate (μmol of substrate cleaved/min) was calculated using linear regression analysis (GraphPad Prism software version 8.01). The reaction rates calculated were used to generate the enzyme kinetic parameters Vmax and Km through non-linear regression analysis (GraphPad Prism software, version 8.01; Michaelis-Menten equation). Where indicated, enzyme activity in the presence of peptide was expressed as a % of the enzyme alone. Data were expressed as mean±SEM and statistical significance was determined using t-tests or one-way ANOVAs followed by Tukey's post-hoc test. P<0.05 was considered as statistically significant. In all assays conducted using 96-well plates, the reaction mixtures in each well were considered an independent experiment.

### Example 3—Effect of Peptides on NEP and ECE1 Activity

SEQ ID NO:9 has been shown to directly increase the activity of both NEP and ECE1. SEQ ID NO:9 is a 20-amino acid residue peptide from the N-terminal domain of myotoxin II derived from the venom of the Central American pit viper Bothrops aspen (Smith A I et al., (2016) Scientific reports 6:22413). A peptide library of SEQ ID NO:9 truncates was prepared by sequentially deleting two amino acids at a time from the C-terminus to provide in order SEQ ID NOS: 10, 11, 12, 5, 1, 13, 14 and 15.

SEQ ID NOS: 1, 5 and 9-15 were screened for their effects on NEP and ECE1 using a high-throughput QFS assay using the method in Example 2 and the following procedure. NEP or ECE1 (0.05 ng/μL) was incubated with each peptide (10 ng/μL) for 1 h at 37° C.

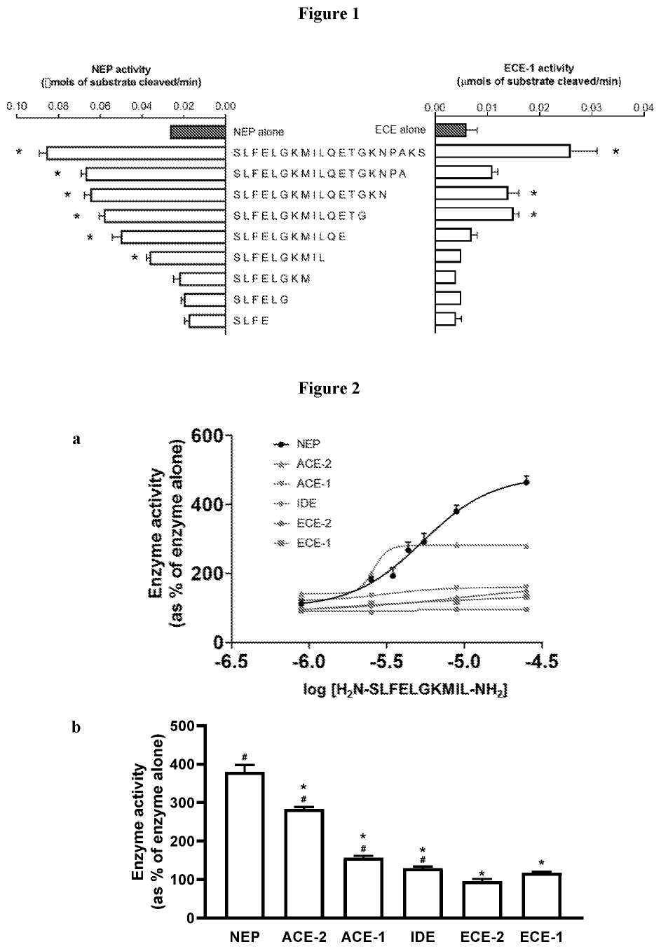

The results are shown in FIG. 1. SEQ ID NO:9 was found to significantly stimulate both NEP and ECE1 activity in line with previous studies. Sequential deletion of amino acids from the C-terminus led to a gradual decrease in the level of NEP stimulation. SEQ ID NOS: 11 and 12 were found to significantly stimulate both NEP and ECE1. SEQ ID NOS: 1 and 5 were found to induce a significant increase in NEP activity while having no effect on the activity of ECE1.

### Example 4—Peptide Stability Study

SEQ ID NO:2 was prepared as a C-terminal amidated analogue of SEQ ID NO:1. The stability of SEQ ID NO:1 and SEQ ID NO:2 was assessed in HEK293 and EA.hy926 cells grown in culture and their break down was monitored by LCMS over 60 min using the following procedure.

HEK293 (passage 24) or Ea.hy926 (passage 37) were seeded into tissue-culture-grade 96-well plates at a density of 20,000 cells/well. After 24 hr, cells were washed with PBS and replaced with Opti-MEM reduced serum media containing SEQ ID NO:1 or SEQ ID NO:2 (9 μM). Aliquots of equal volume were taken at T=0 and 60 min. SEQ ID NO:2 was added to control wells having media (no cells) only. Samples were immediately acidified in 0.1% TFA, snap frozen in dry ice and stored in −80° C. until analysed by LCMS.

Samples were analyzed by LC-MS/MS on a Shimadzu Nexera uHPLC (Japan) coupled to a Triple Tof 5600 mass spectrometer (ABSCIEX, Canada) equipped with a duo electrospray ion source. One μL of each extract was injected onto a 2.1 mm×100 mm Zorbax C18 1.8 um column (Agilent) at 200 μL/min. Linear gradients of 1-50% solvent B over 7 min at 200 uL/min flow rate, followed by a steeper gradient from 50% to 98% solvent B in 1 min were used for peptide elution. Solvent B was held at 98% for 3 min for washing the column and returned to 1% solvent B for equilibration prior to the next sample injection. Solvent A consisted of 0.1% formic acid (aq) and solvent B contained 90/10 acetonitrile/0.1% formic acid (aq). The ionspray voltage was set to 5500 V, declustering potential (DP) 100 V, curtain gas flow 25, nebuliser gas 1 (GS1) 50, GS2 to 60, interface heater at 150° C. and the turbo heater to 500° C. The mass spectrometer acquired 500 ms full scan high resolution, at 30,000 resolving power, TOF-MS data over the mass range m/z 600 to 1200. SEQ ID NO:1 and SEQ ID NO:2 were quantified using the [M+2E1]⁺ doubly charged molecular ion from m/z 510.270 to 510.325. The data was acquired using Analyst TF 1.6 and quantification was carried out using MultiQuant 2.1.1 software (ABSCIEX, Canada).

The relative amount of SEQ ID NO:2 remaining in the medium of endothelial cells at 60 min (26%±5%) was not significantly different compared with SEQ ID NO:1 (23%±2.4%). However, the amount of SEQ ID NO:2 remaining in the medium of HEK293 cells (54%±4% as % of initial) was significantly higher compared with SEQ ID NO:1 (29%±3% as % of initial). After 60 min, 63%±14% of SEQ ID NO:2 remained in the medium with no cells indicating possible adherence to plastic. The results indicate that SEQ ID NO:2 may be more biologically stable than SEQ ID NO:1. It is noted that the differences in stability of SEQ ID NO:2 observed for HEK293 and Ea.hy926 cells may be due to differences in cell surface proteases between the two cell types.

### Example 5—NEP and ACE2 Enzyme Kinetics Study

The effect of SEQ ID NO:2 on enzyme kinetic parameters vₘₐₓ and K_{M} of NEP and ACE2 was determined by monitoring the cleavage of a QFS by rhNEP and rhACE2, respectively, using the method in Example 2 and the following procedures. rhNEP (0.05 μg/μL) or rhACE2 (0.1 ng/μL) was incubated with SEQ ID NO:2 for 1 h at 37° C. Enzyme controls included the relevant buffer only. The reaction was started by the addition of the relevant QFS (NEP QFS at 0-120 μM final concentration, ACE2 QFS at 0-100 μM final concentration).

The vₘₐₓ of rhNEP in the presence of SEQ ID NO:2 (0.035±0.002 μmol substrate cleaved/min) was significantly higher compared with enzyme alone (0.008±0.001 μmol substrate cleaved/min). This may indicate that SEQ ID NO:2 enhances the rate of substrate cleavage by NEP. The K_{M} of rhNEP in the presence of SEQ ID NO:2 (8.7±0.8 μM) was not significantly different compared with NEP (12.13±0.8 μM) alone.

The vₘₐₓ of rhACE2 in the presence of SEQ ID NO:2 (0.09±0.002 μmol substrate cleaved/min) was significantly higher than rhACE2 alone (0.05±0.001 μmol substrate cleaved/min; P<0.0001, unpaired t-test; n=4-5). This may indicate that SEQ ID NO:2 enhances the rate of substrate cleavage by ACE2. The K_{M} of rhACE2 in the presence of SEQ ID NO:2 (17±1.4 μM) was also significantly higher than rhACE2 alone (8±0.6 JIM).

### Example 6—Effect of SEQ ID NO:2 on the Activity of Aβ-Degrading Enzymes

SEQ ID NO:2 was assessed in a QFS assay against enzymes NEP, ECE1, ECE2, ACE1, ACE2, IDE and MMP-2 using the method in Example 2 and the following procedure. NEP, ACE1, ECE2 (0.05 ng/μL), ACE2, IDE (0.10 ng/μL), and ECE1 (6.4 ng/μL) were incubated with SEQ ID NO:2 (0.9-26 μM) or scrambled control (9 μM) for 1 h at 37° C. Prior to use, MMP2 (0.50 ng/μL) was activated using p-aminophenylmercuric acetate to a final concentration of 100 μM for 1 h at 37° C. Following activation, MMP2 was incubated with SEQ ID NO:2 for 1 h at 37° C. before adding the appropriate QFS.

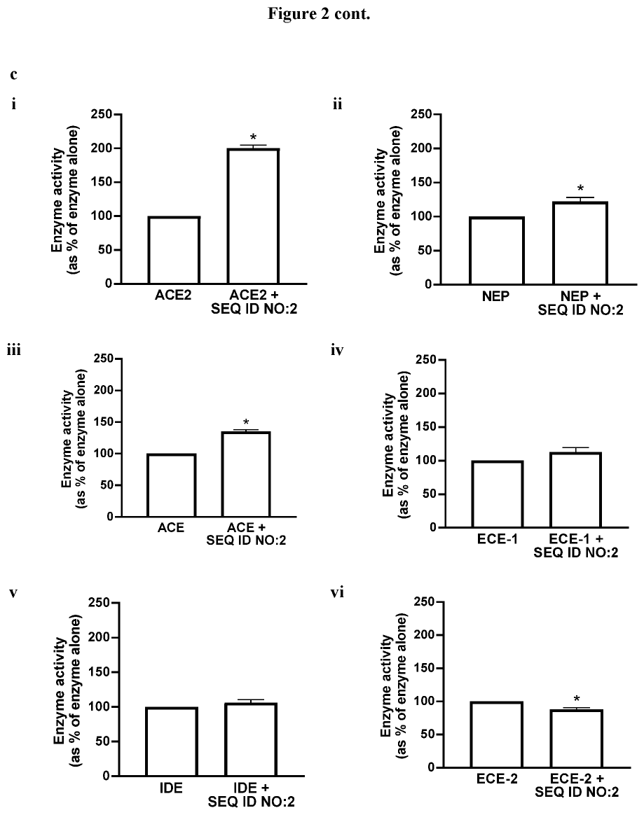

The enzyme activity data for NEP, ECE1, ECE2, ACE1 and ACE2 are illustrated in FIG. 2. SEQ ID NO:2 increased the activity of NEP, ACE2, ACE1 and IDE in a concentration-dependent manner, but did not affect the activity of ECE1 or ECE2 (FIG. 2a). The submaximal concentration of SEQ ID NO:2 against NEP was 9 μM.

FIG. 2b shows enzyme activity in the presence of 9 μM SEQ ID NO:2. At this concentration of SEQ ID NO:2, the maximum level of SEQ ID NO:2 induced stimulation of NEP was greater than that of ACE2, the activity of NEP and ACE2 in the presence of SEQ ID NO:2 being 380% and 283%, respectively, of enzyme alone controls. The activity of ACE1 and IDE was significantly less compared with ACE2 and NEP in the presence of SEQ ID NO:2. The activity of all proteases except ECE1 and ECE2 were significantly higher in the presence of SEQ ID NO:2, compared with respective enzyme alone. SEQ ID NO:2 did not affect the activity of MMP-2 (not shown in FIG. 2b). There was no significant difference in the activity of MMP-2 in the presence (0.7±0.2 μmol of substrate cleaved) or absence (0.8±0.1 μmol of substrate cleaved) of SEQ ID NO:2.

FIG. 2c shows enzyme activity in the presence of 2 μM SEQ ID NO:2. At this concentration of SEQ ID NO:2, the activity of ACE2, ACE1 and NEP was significantly increased in the presence of SEQ ID NO:2 compared with respective enzyme alone (FIG. 2c (i), (ii) and (iii)). The presence of SEQ ID NO:2 did not increase the activity of ECE1, IDE or ECE2 compared with respective enzyme alone (FIG. 2c(iv), (v) and (vi)).

### Example 7—Cleavage of Synthetic Aβ40 and Aβ42 by NEP

The effect of SEQ ID NO:2 on the breakdown of synthetic Aβ40 and Aβ42 by NEP was assessed using the following procedure.

Synthetic Aβ40 (0.10 μg/μL) was added to a reaction mixture containing NEP (0.05 ng/μL) pre-incubated with SEQ ID NO:2 or scrambled control (9 μM) for 1 h at 37° C. Synthetic Aβ42 (0.05 μg/μL) was added to a reaction mixture containing NEP (0.15 ng/μL) preincubated with either scrambled control or SEQ ID NO:2 (9 μM) for 1 h at 37° C. Samples containing enzyme alone had only NEP buffer (Table 2). The cleavage reaction was allowed to continue in a thermomixer at 37° C. Aliquots of equal volume were taken over 24 h. Aliquots were immediately acidified with 0.1% trifluoroacetic acid (TFA) to terminate enzyme activity and snap frozen in dry ice. Samples were stored at −80° C. until analysed by LCMS.

Samples were analysed by LC-MS/MS using a quadrupole TOF mass spectrometer (MicroTOFq, Bruker Daltonics, Bremen, Germany) coupled online with a 1200 series nano-HPLC (Agilent technologies, Santa Clara, CA, USA). Samples were injected onto a zorbax 300SB reversed phase trap column with 95% solvent A (0.1% formic acid) at a flow rate of 10 μL/minute. The peptides were eluted over a 10-min gradient to 70% solvent B (80% acetonitrile, 0.1% formic acid) and separated on a 15-cm, 75-μmID zorbax 300SB nanocolumn. The eluent was nebulised and ionised using the Bruker nano-ESI source with a capillary voltage of 4500 V, dry gas at 180° C., flow rate of 5 L/minute and nebuliser gas pressure at 300 mbar. The MS acquisition was in selected ion monitoring mode after selected ion extraction of the MS spectra. Prior to analysis, the qTOF mass spectrometer was calibrated using a 1:50 dilution tuning mix (Agilent technologies, Santa Clara, CA, USA). Data from MS run were processed in Skyline version 19.1.0.193 (Uni. Of Washington, WA, USA) to perform ion chromatogram extractions and peak integrations.

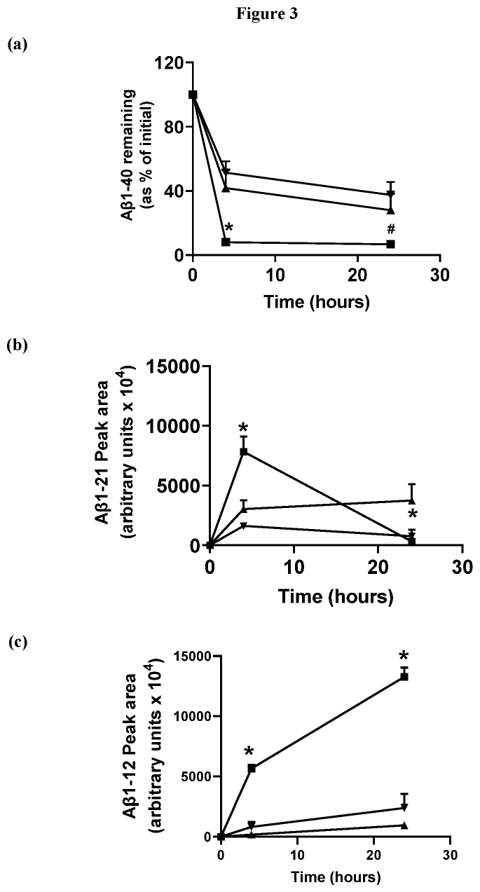

Cleavage of synthetic APO: SEQ ID NO:2 enhanced the cleavage of synthetic Aβ40 by NEP. The amount of Aβ40 remaining (as % of initial) over time for each sample is shown in FIG. 3a (NEP alone—down triangles; NEP+scrambled control—up triangles; NEP+SEQ ID NO:2—squares). Peak area (measured in arbitrary units) corresponding to each peptide was taken as the relative amount present at each time point. The amount of Aβ40 remaining after 4 h in the presence of SEQ ID NO:2 (1.3×10⁶±2.7×10⁵) was significantly less compared with NEP alone (3.2×10⁶±2.9×10⁵) or scrambled control (6.4×10⁶±1.7×10⁶). Breakdown of Aβ40 in the presence of SEQ ID NO:2 was complete after 4 h. However, Aβ40 cleavage continued over the next 20 h in the presence of scrambled control and NEP alone. There was no significant difference in the amount of Aβ40 remaining after 24 h in the presence of SEQ ID NO:2 or scrambled control.

Analysis by LCMS identified the following two N-terminal fragments of Aβ1-Aβ1-21 and Aβ1-12. The levels of Aβ1-21 detected for each sample over time are shown in FIG. 3b (NEP alone—down triangles; NEP+scrambled control—up triangles; NEP+SEQ ID NO:2—squares). Levels of Aβ1-21 increased between 0 and 4 h (7.9×10⁷±1.3×10⁷, P=0.03; significantly different compared with scrambled control at 4 h) but decreased between 4 and 24 h (4.1×10⁶±4.8×10⁵ at 24 h; P=0.03; significantly different compared with scrambled control). An initial increase followed by a decrease in Aβ1-21 levels was also observed with NEP alone, but not the scrambled control.

The levels of Aβ1-12 detected for each sample over time are shown in FIG. 3c (NEP alone—down triangles; NEP+scrambled control—up triangles; NEP+SEQ ID NO:2—squares). Levels of Aβ1-12 increased over time under each treatment. After 24 h, peak area corresponding to Aβ1-12 in the presence of SEQ ID NO:2 (1.3×10⁸±7.9×10⁶) was significantly higher compared with scrambled control (9.7×10⁶±2.1×10⁶). It is noted that for SEQ ID NO:2 samples the increase in Aβ1-12 peak area over 4-20 h (7.6×7±5.7×10⁶) corresponded to a decrease in that of Aβ1-21 (7.5×10⁷±1.3×10⁷) over the same time period, which may indicate possible secondary cleavage of Aβ1-21 by NEP to produce Aβ1-12. A decrease in Aβ1-21 and increase in Aβ1-12 was not observed in the presence of the scrambled control, which may reflect the lower efficiency of NEP in the presence of the scrambled control.

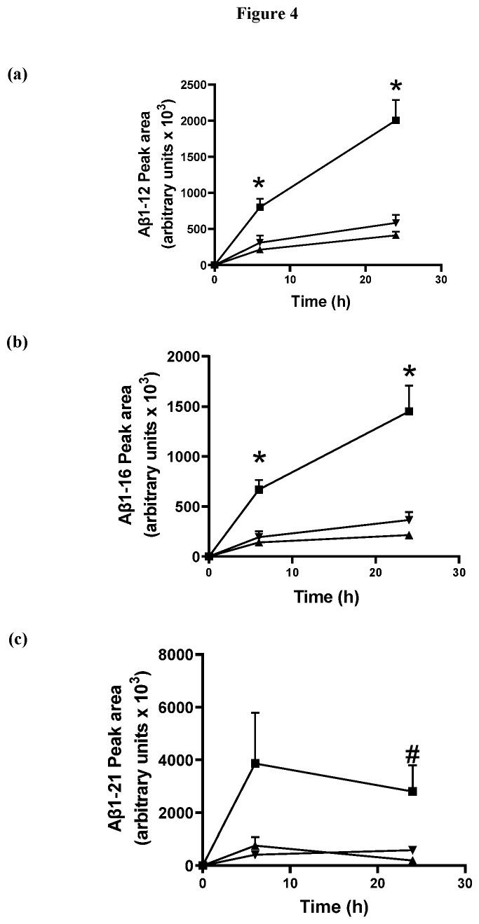

Cleavage of synthetic Aβ42: Monitoring the breakdown on Aβ42 by LCMS led to the detection of the following N-terminal cleavage fragments: Aβ1-12, Aβ1-16 and Aβ1-21. The levels of Aβ1-12, Aβ1-16 and Aβ1-21 detected for each sample over time are shown FIGS. 4a-c respectively (NEP alone—circles; NEP+scrambled control—triangles; NEP+SEQ ID NO:2—squares). Levels of all three cleavage products increased over 24 h under each treatment. However, the amount formed was significantly higher in the presence of SEQ ID NO:2 compared with scrambled control indicating enhanced NEP mediated breakdown of Aβ1-42 in the presence of SEQ ID NO:2. These data suggest that SEQ ID NO:2 may increase the rate of NEP-mediated cleavage of synthetic Aβ42. Given the widely described toxic effects Aβ42, these results may provide an indication that SEQ ID NO:2 could be used to manipulate NEP activity in the setting of Alzheimer's Disease.

### Example 8—Cleavage of Synthetic Ang II by ACE2

The effect of SEQ ID NO:2 on the breakdown of synthetic Ang II to Ang 1-7 by ACE2 was assessed using the following procedure.

rhACE2 (0.1 ng/μL) was incubated with SEQ ID NO:2 (1.7 μM) for 1 h at 37° C. Ang II (0.02 μg/μL) was then added to the reaction mixture. Aliquots of equal volume were collected at 0, 3, 6 and 24 h. Aliquots were immediately acidified with 0.1% TFA. Samples were snap frozen in dry ice and lyophilized for analysis by LCMS. The samples were analysed by LC-MS/MS using the method in Example 7.

ACE2-mediated breakdown of Ang II over 24 h was significantly greater in the presence of SEQ ID NO:2 compared with enzyme alone (decrease in peak area of 83±5 and 58±4% of initial respectively; P<0.01, unpaired t-test, n=4). The production of Ang 1-7 in the presence of SEQ ID NO:2 (165±12% of ACE2 alone) was significantly higher compared to ACE2 alone (P<0.01, unpaired t-test, n=4). Given that Ang 1-7 is known to have cardio- and reno-protective effects, these results may provide an indication that SEQ ID NO:2 could be used in the treatment of cardiovascular and renal diseases.

### Example 9—Effect of SEQ ID NO:2 on Ang II-Induced Expression of IL-6 and Collagen III

The effect of SEQ ID NO:2 on Ang II-induced expression of inflammatory marker IL-6 and fibrosis marker Collagen III in EA.hy926 cells was assessed by western blotting using the following procedure.

EA.hy926 cells (passage 40) were seeded at a density of 1×10⁶ cells/mL. After 24 hours, cells were incubated overnight in reduced serum media. Cells were then treated over 24 h as follows:
1. Vehicle control
2. Ang II (10 nM)
3. Ang II (10 nM)+SEQ ID NO:1 (26 μM)
4. Ang II (10 nM)+rhACE2 (0.05 ng/μL)
5. Ang II (10 nM)+SEQ ID NO:1 (26 μM)+rhACE2 (0.05 ng/μL)+ACE2 inhibitor (MLN-4760; 10 μM)

Following incubation, cells were lysed using lysis buffer (Thermo Fisher Scientific, Cat #89900) containing 1% protease inhibitor (Thermo Fisher Scientific, Cat #1861278). Cell lysate was centrifuged at 13,000 rpm for 10 min. Protein concentration in the supernatant was determined by NanoDrop (Thermo Fisher Scientific, Cat #ND-1000). Supernatants were stored at −30° C. until analysed.

Samples were diluted in SDS-PAGE reducing sample buffer (Bio-Rad Laboratories, Cat #1610747; California, USA) containing 1% β-mercaptoethanol (Bio-Rad Laboratories, Cat #221610710) to reach a final concentration of 30 μg/μL. Prior to loading, samples were heated at 95° C. for 5 min. Samples were loaded into the wells alongside the molecular weight marker (Life Technologies, Cat #LC5800; California, USA). Electrophoresis was conducted at 200 V for 40 min in 1× running buffer. Following protein separation by SDS-PAGE, proteins were blotted onto nitrocellulose membranes at 100 V for 1 h in 1× transfer buffer. The membranes were then blocked in 5% skim milk in 1× Tris-buffered saline (TBST) containing 0.05% Tween 20 (Thermo Fisher Scientific, Cat #28352) for 1 h at room temperature. The membranes were incubated with IL-6 (1:500; Thermo Fisher Scientific, Cat #700480) or collagen III (1:500; Invitrogen, Cat #PA5-27828) primary antibodies while rocking at 4° C. overnight. Membranes were then probed with secondary antibody conjugated with horseradish peroxidase (HRP) (1:6000) for 1 h at room temperature. The HRP-labelled proteins were detected using chemiluminescence SuperSignal™ ECL Western Blotting Substrate (Thermo Fisher Scientific, Cat #34577) at 1:1 ratio. Beta-actin was used as a loading control. Protein bands were analysed using ImageJ (version 1.51) and band density was normalised with the respective beta-actin.

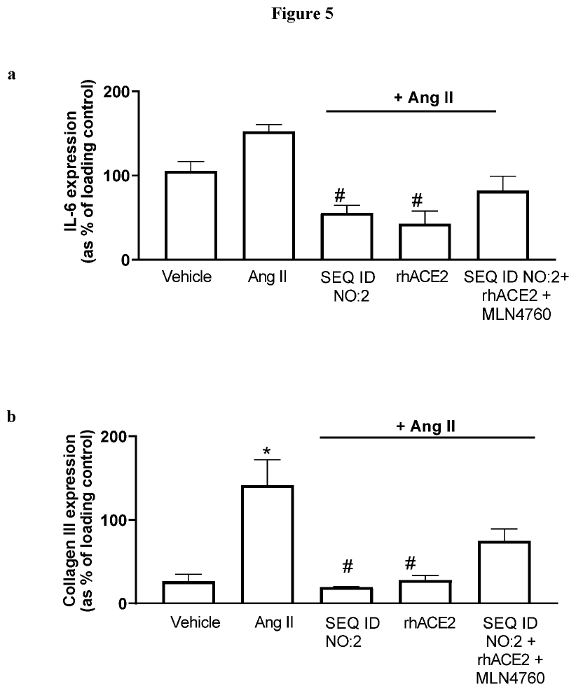

IL-6 expression is shown in FIG. 5a. IL-6 expression in cells treated with Ang II (10 nM) was significantly higher (152±8%) compared with vehicle treatment (105±11%). In cells treated with SEQ ID NO:2 (26 μM) and/or rhACE2 (0.05 ng/μL), IL-6 expression was significantly lower compared to cells treated with Ang II alone (P<0.05, unpaired t-test, n=3). There was no difference in the expression of IL-6 in the presence of MLN-4760.

Collagen III expression is shown in FIG. 5b. Collagen III expression in cells treated with Ang II was significantly higher (141±30%) compared with vehicle treatment (26±9%) (P=0.02). Collagen III expression was significantly lower in cells treated with 2A and/or ACE2 compared to cells treated with Ang II alone (P<0.05, unpaired t-test, n=3). Collagen III expression in the presence of ACE2 inhibitor MLN-4760 (75±14%) was significantly higher than that of cells treated with Ang II and 2A (28±6%; P=0.02).

### Example 10—Effect of Pre-Treatment SEQ ID NO:2 on SARS-CoV-2 Infection

The effect of pre-treatment with SEQ ID NO:2 on SARS-CoV-2 infection in Vero cells was assessed using the following procedure.

Vero cells were grown to confluency in 24 well plates. The cells were treated for one hour with SEQ ID NO:2 or PBS. SEQ ID NO:2 was then removed and the cells were placed in Dulbecco's Modified Eagle's medium (DMEM)+penicillin/streptomycin+2% fetal calf serum (FCS). Cells were then infected for 1 h with 0.5×10\hat{ }4 plaque-forming units (PFU) of SARS-CoV-2 at 37° C. SARS-CoV-2 was removed and the media was replaced with DMEM+penicillin/streptomycin+2% FCS and SEQ ID NO:2 or PBS. After 8 h, supernatant was collected to determine viral titre. Cells were lysed in Buffer RLT (Qiagen) and RNA was extracted using the Qiagen RNAEasy Plus kit. cDNA was synthesised and SYBR Green qPCR was performed for SARS-CoV-2 mpro, host GAPDH, IL-6 and IL8.

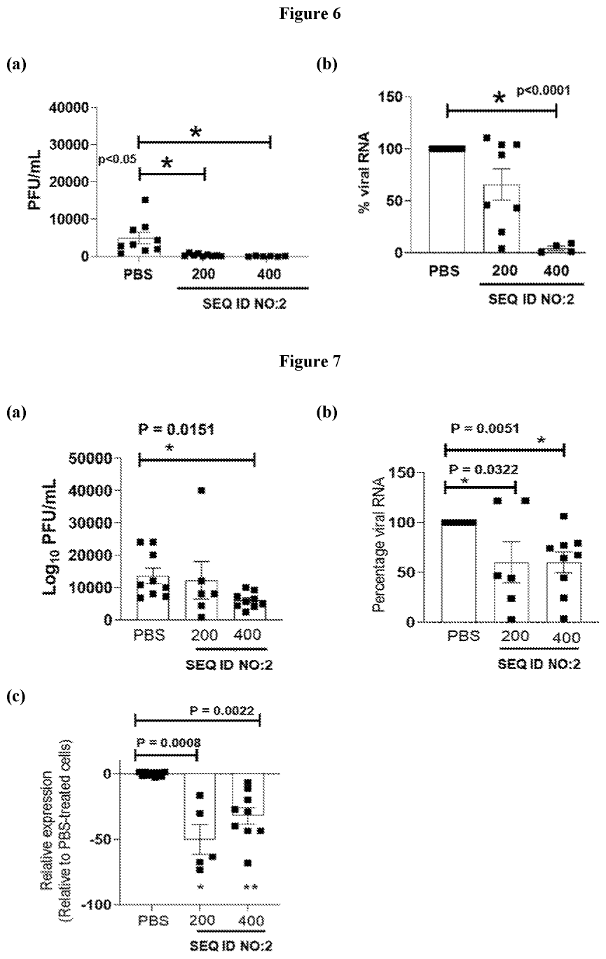

The pre-treatment results are shown in FIG. 6. FIG. 6a shows that presence of 200 ng/μL and 400 ng/μL SEQ ID NO:2 significantly reduced viral titre compared to PBS (P<0.05 by one-way ANOVA). FIG. 6b shows that presence of 400 ng/μL SEQ ID NO:2 significantly reduced viral RNA compared to PBS (P<0.001 by one sample t-test). These results may provide an indication that SEQ ID NO:2 could be used in the prevention of SARS-CoV-2 infection.

### Example 11—Effect of Post-Treatment with SEQ ID NO:2 on SARS-CoV-2 Infection and Expression of IL-6

The effect of post-treatment with SEQ ID NO:2 on SARS-CoV-2 infection and IL-6 expression in Vero cells was assessed using the following procedure.

Vero cells were grown to confluency in 24 well plates. The media was removed and the cells were placed in DMEM+penicillin/streptomycin+2% FCS. The cells were then infected for 1 h with 0.5×10\hat{ }4 PFU of SARS-CoV-2 at 37° C. SARS-CoV-2 was removed and the media was replaced with DMEM+penicillin/streptomycin+2% FCS. After 8 h, SEQ ID NO:2 or PBS was added to the cell culture supernatant. After 8 h, supernatant was collected to determine viral titre. Cells were lysed in Buffer RLT (Qiagen) and RNA was extracted using the Qiagen RNAEasy Plus kit. cDNA was synthesised and SYBR Green qPCR was performed for SARS-CoV-2 mpro, host GAPDH, IL-6 and IL8.

The post-treatment results are shown in FIG. 7. FIG. 7a shows that presence of 400 ng/μL SEQ ID NO:2 significantly reduced viral titre compared to PBS (P=0.0151 by Kruskal-Wallis test). FIG. 7b shows that presence of 200 ng/μL and 400 ng/μL SEQ ID NO:2 significantly reduced viral RNA compared to PBS (P=0.0322 and P=0.0051, respectively, by one sample t-test). These results may provide an indication that SEQ ID NO:2 could be used in the treatment of SARS-CoV-2 infection. FIG. 7c shows that presence of 200 ng/μL and 400 ng/μL SEQ ID NO:2 significantly reduced IL-6 expression compared to PBS (P=0.0008 and P=0.0022, respectively, by Kruskal-Wallis test). This result may provide an indication that SEQ ID NO:2 could be used in the treatment of SARS-CoV-2-associated inflammation.

### Example 12—Effects of Peptide SEQ ID NO:2 on IL-6 Expression and Collagen I Expression in STZ Diabetic Mouse Model

The following study was undertaken to assess the effects of peptide SEQ ID NO: 2 on inflammation and fibrosis in a STZ diabetic mouse model.

Mice were divided into two groups, all mice were treated with STZ (150 mg/kg) 1 week before starting treatment. Prior to STZ treatment, urine and blood samples were taken and a blood glucose test undertaken. Similarly, before treatment began, one week post STZ administration, urine and blood samples were taken and blood glucose was assessed. The two treatment groups were then treated with either vehicle only or SEQ ID NO: 2 (1 mg/kg) subcutaneously by minipump. The mice were then monitored for 4 weeks before sacrifice. Monitoring included assessing the weight of each animal every day, and taking urine and blood samples and measuring blood glucose at 2 weeks post treatment and immediately before sacrifice.

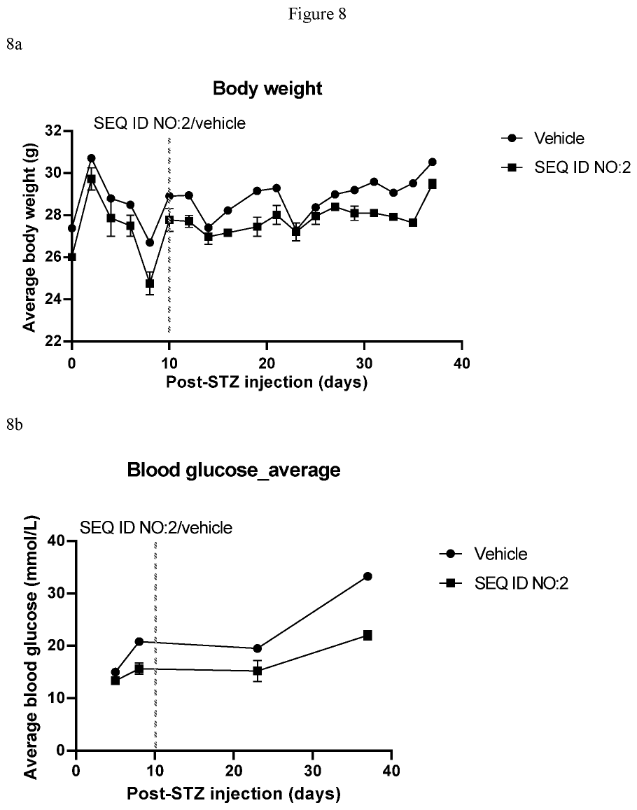

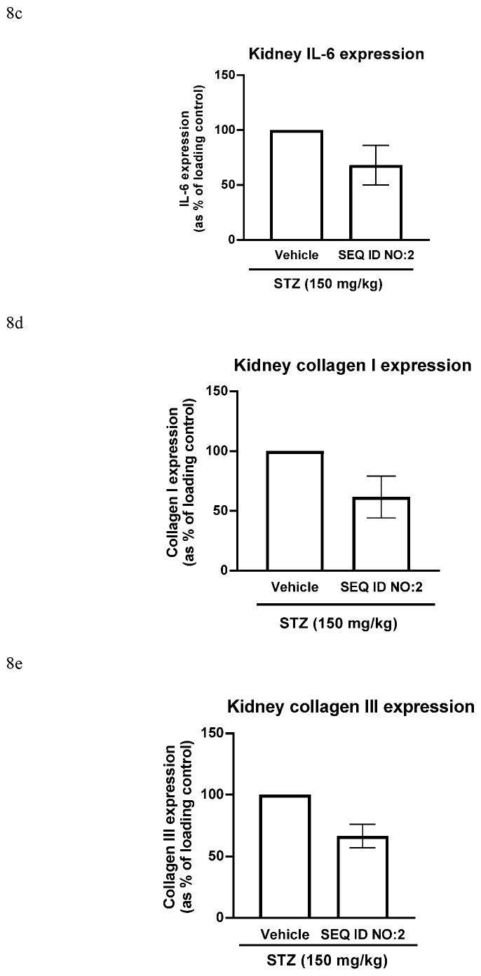

The results are shown in FIG. 8. The body weight of the animals treated with vehicle and SEQ ID NO:2 followed similar trends but the body weight of the peptide treated mice was slightly lower than the vehicle treated mice (FIG. 8a). The blood glucose of the mice treated with SEQ ID NO:2 was lower than the blood glucose of the mice treated with vehicle (FIG. 8b). The kidney IL-6 expression (FIG. 8c), collagen I expression (FIG. 8d) and collagen III expression (FIG. 8e) in the mice treated with SEQ ID NO:2 was lower than those of the vehicle treated mice indicating that SEQ ID NO:2 reduced the incidence of kidney inflammation and fibrosis in diabetic mice.

### Example 13—Mouse Study Design for AD Model

The following is a study is for assessing the effect of SEQ ID NO:2 in a mouse model of Alzheimer's Disease (AD).

Study design: The study involves the following treatment groups (n=12 per group):
Group 1: SEQ ID NO:2 administered via subcutaneous osmotic mini-pumps
Group 2: SEQ ID NO:2+L-arginine (500 mg/kg) administered via subcutaneous osmotic mini-pumps
Group 3: SEQ ID NO:2+L-arginine (1000 mg/kg) administered via subcutaneous osmotic mini-pumps
Group 4: Direct brain infusion of SEQ ID NO:2

Each treatment group will include 12 mice to account for possible premature death and achieve statistical power. In each group the mini-pumps replaced every 4 weeks. Male B6C₃-Tg (APPswe,PSEN1dE9)85Dbo mice (Jackson Laboratories) will be used which develop Aβ plaques at 6-7 months of age. In APP transgenic mouse models, administration of a drug lead prior to plaque formation is expected to delay the rate of amyloid deposition (Karran, E. and Hardy, J. (2014) Ann Neurol 76, 185-205). Therefore, drug infusions will begin at the age of 5 months and continue for 8 weeks to offer the best chance of stimulating NEP and therefore delaying or preventing plaque formation. Tissues will be harvested at the age of 9 months. Intrinsic variation in plaque size is known to be minimal at this age and thus is expected to have a negligible effect on data analysis.

In Groups 1-3 above, SEQ ID NO:2 will be administered to mice over 8 weeks subcutaneously via osmotic mini-pumps. This timing and duration of treatment is expected to provide the best chance of achieving a stable concentration in plasma and therefore entering the brain. The mice in Group 4 above will be anaesthetised and a cannula inserted into the left lateral ventricle based on stereotactic coordinates. SEQ ID NO:2 will be delivered to the lateral ventricle using an implantable Alzet Osmotic Mini Pump and Mouse Brain Infusion Kit #3.

Behavioural studies: At the end of the 8-week treatment period, the mice will be subjected to the radial arm maze test which is well known in the art.

Tissue processing: After behavioural testing, the mice will be killed by an overdose of pentobarbitone (100 mg/kg) and their brains harvested, processed and embedded in paraffin. One hemisphere will be used to determine plaque load and other half to quantitate Aβ levels as well as for the presence of SEQ ID NO:2.

Immunostaining to determine plaque load: The distribution and extent of neuropathological changes will be correlated with Aβ plaque load, which will be determined by immunostaining with antibodies directed against Aβ as well as staining fibrillar amyloid deposits with Thioflavin S. Immunoreactivity will be assessed in both hippocampal and cortical regions by the relative staining intensity per mm².

Quantitation of Aβ levels: Brain tissue will be homogenized in PBS in the presence of a cocktail of protease inhibitors to minimize proteolytic degradation. After centrifugation (100,000 g, 30 min) the soluble fraction will be resolved on Tricine SDS-PAGE and detected by Western blotting using anti-Aβ antibodies. The level of chemiluminescence will be quantified with respect to a known amount of β-actin.

Detecting the presence of SEQ ID NO:2: Presence of SEQ ID NO:2 in the brain will be determined by subjecting brain tissue homogenates to analysis by advanced proteomic techniques. First, a bioanalytical method with sufficient sensitivity to detect SEQ ID NO:2 in brain tissue homogenates and plasma will be developed and validated. This can then be applied to detect and quantitate the levels of SEQ ID NO:2 in brain tissues and plasma obtained from the above listed treatment groups. Plasma/brain ratio of the drug lead will be determined using the data generated.

It is to be understood that, if any prior art publication is referred to herein, such reference does not constitute an admission that the publication forms a part of the common general knowledge in the art, in Australia or any other country.

### Example 14—Kidney Inflammation and Fibrosis in a STZ Diabetic Mouse Model

The following study was undertaken to assess the effects of peptide SEQ ID NO: 2 on kidney inflammation and fibrosis in a STZ diabetic mouse model.

Mice were divided into four groups, two groups of mice remained untreated (ND) and two groups of mice were treated with STZ (150 mg/kg) by i.p. injection 1 week before starting treatment with SEQ ID NO. 2 or vehicle. Before treatment with SEQ ID NO. 2 or vehicle began, one week post STZ administration, 24 hour urine and blood samples were taken and blood glucose was assessed in all mice. The two ND groups and the two STZ treatment groups were then treated with either vehicle (10% DMSO/PBS) only or SEQ ID NO: 2 (1 mg/kg) subcutaneously by osmotic minipump. The mice were then monitored for 3 months before sacrifice. Monitoring included assessing the weight of each animal every day, taking blood samples biweekly and 24 hour urine samples at 1, 2 and 3 months post treatment. 24 hour urine samples were collected from mice placed in metabolic cages. At 3 months after treatment, a blood sample was collected (cardiac bleed) and the animals were sacrificed. Kidney and heart tissues were collected and weight and the tissues processed for in vitro analysis.

Urine samples were analysed for urinary albumin ELISA. The volume of urine was measured and then samples were diluted by 1:8000 with water. The samples were incubated for 1 h in antibody-coated 96-well plates (in duplicate). After incubation, a Development solution (100 μL) containing sandwich antibody was added to the wells and the samples incubated for 10 minutes in the dark. Stop solution (100 μL) was added and the optical density was measured at 450 nm.

The kidney tissue samples were analysed for fibrosis using Masson's trichrome staining. 4 μm-thick paraffin-embedded kidneys were sectioned. After dewaxing, sections were post-fixed in Bouin's fixative overnight. The slides were stained in Weigert's iron haematoxylin followed by Biebrich scarlet-acid fuchsin solution. The slides were differentiated in phosphomolybdic-phosphotungstic acid solution and then further stained in aniline blue solution. After differentiation in 1% acetic acid solution the slides were mounted with mounting medium and the sections were imaged on an Aperio Slide Scanner at ×20 magnification. The images were analysed on ImageJ.

Kidney samples in mice sacrificed one month after treatment were also analysed for the presence of IL-6 by western blot analysis. Homogenised kidney samples were heat-denatured and separated using SDS-PAGE. The membranes were incubated with IL-6 primary antibody at 1:500 dilution, followed by incubation with a secondary antibody (goad anti-rabbit antibody at 1:16000) and developed the following day using enhanced chemiluminescence (ECL) as a substrate. β-actin was used as a loading control.

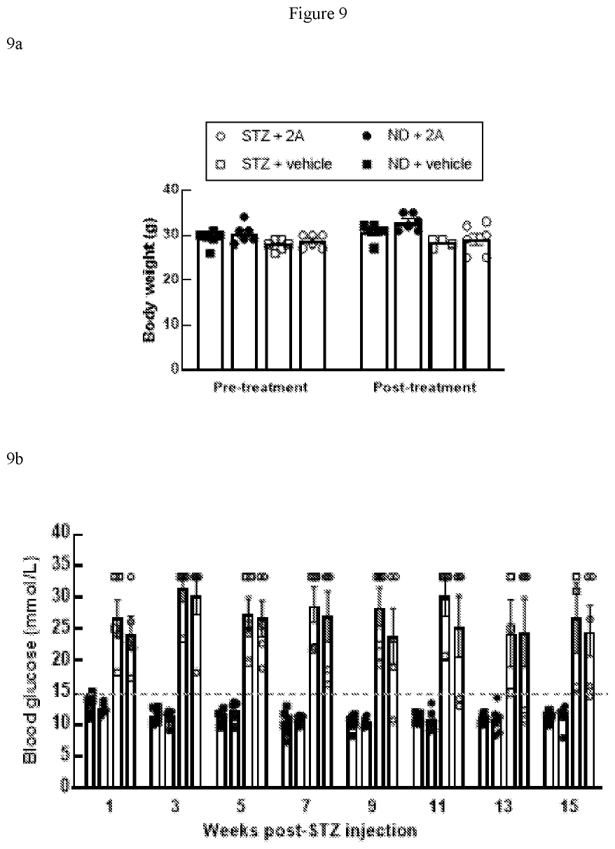

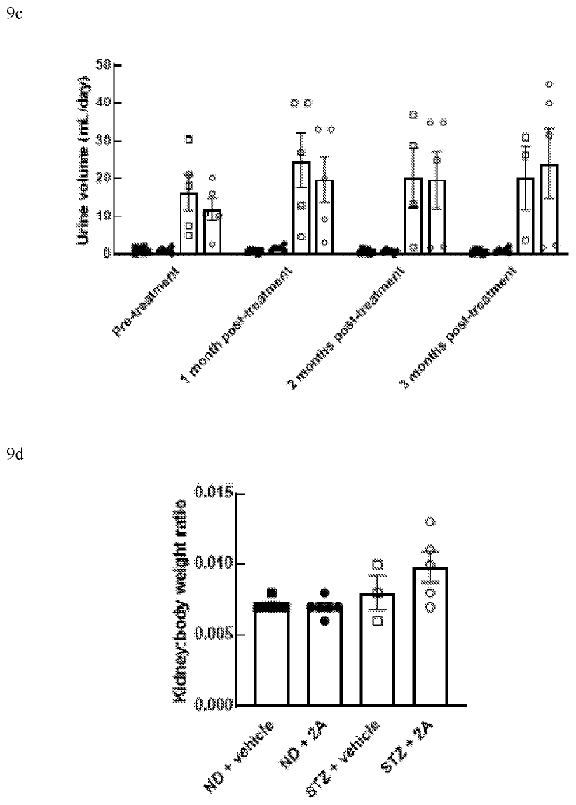

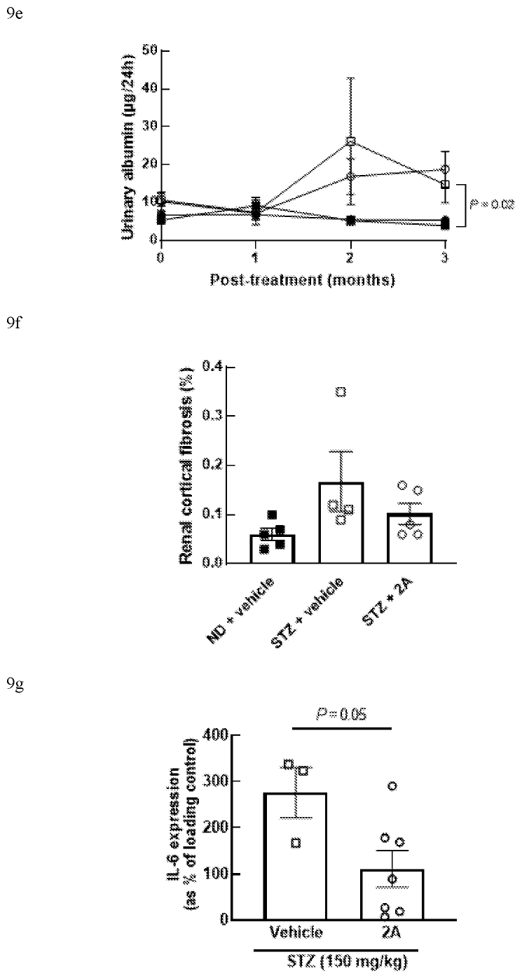

The results are shown in FIG. 9 (SEQ ID NO. 2 is referred to as 2A in FIG. 9). The body weight of the ND and the STZ animals treated with vehicle and SEQ ID NO:2 followed similar trends before and after treatment with SEQ ID NO. 2 (2A) or vehicle (FIG. 9a). The post treatment blood glucose of the ND mice was similar whether treated with SEQ ID NO. 2 (2A) or vehicle whereas in STZ mice treated with SEQ ID NO:2 (2A) the blood glucose was marginally lower than the blood glucose of the STZ mice treated with vehicle (FIG. 9b). The urine volume per day of ND mice before and after treatment with either SEQ ID NO. 2 (2A) or vehicle was small and was the same before or after treatment in contrast to the STZ mice which had an increased urine volume per day whether treated with SEQ ID NO. 2 (2A) or vehicle (FIG. 9c). The kidney:bodyweight ratio remained the same in ND mice whether treated with SEQ ID NO. 2 (2A) or vehicle but the ratio increased in the STZ mice treated with vehicle and even more in the STZ mice treated with SEQ ID NO. 2 (2A) (FIG. 9d). The urinary albumin remained the same in ND mice whether treated with SEQ ID NO. 2 (2A) or vehicle and while at 1 month, the STZ mice had similar levels of urinary albumin to the ND mice, the urinary albumin increased in the STZ mice at 2 and 3 months post treatment with either vehicle or SEQ ID NO. 2 (2A) (FIG. 9e). Analysis of the kidney tissue for renal cortical fibrosis showed a tendency to be lower in the STZ mice treated with SEQ ID NO. 2 (2A) compared to the STZ mice treated with vehicle, although both STZ treated groups had increased fibroses compared to the ND vehicle treated mice (FIG. 9f). The kidney IL-6 expression (FIG. 8g), in the STZ mice treated with SEQ ID NO:2 (2A) was lower than those of the vehicle treated STZ mice indicating that SEQ ID NO:2 (2A) reduced the incidence of kidney inflammation and has a tendency to reduce kidney fibrosis in diabetic mice.

### Example 15—Effects of SEQ ID NO. 2 in a Mouse Model of Alzheimer's Disease

The following is a study is for assessing the effect of SEQ ID NO:2 in a mouse model of Alzheimer's Disease (AD).

All animal experiments were conducted in strict accordance with the Australian Code of Practice for the Care and Use of Animals for Scientific Purposes. Local approval was obtained from The University of Queensland's Animal Ethics Committee. All wild-type (WT) and transgenic mice (5×FAD tg) used were from the same litters to act as appropriate littermate controls. Thirty-four 2.5-3-month-old female and twenty-five 2.5-3-month-old male mice were used in this study. Animals were housed in groups of 3-5 animals per cage and maintained on a 12 hr light-dark cycle. Food and water were provided ad libitum.

All testing was conducted between 8 AM-6 PM, at approximately the same time each day. Prior to commencing any behavioural testing, mice were habituated to handling and handler smell to minimize handling-related stress. Each mouse was handled daily by the experimenter conducting the behavioural tasks for 30-s to 1-min each day. Handling involved picking up the mouse (by the base of the tail) from the home cage and placing in the palm of a gloved hand. This process was repeated daily, at approximately the same time each day, for a total of 8 days prior to testing. The bedding of the animals' home cage was not changed 2-days prior habituation to handling and for the duration of the behavioural tests.

The open field/activity monitor arena consisted of a clear Perspex chamber (45×45×45 cm). The chamber was equipped with three 16-beam infrared arrays (Med Associates Inc., USA) housed within a sound attenuated box containing a ventilation fan. The chamber was designed to contain two pre-defined zones: a ‘centre’ and ‘outside’ zone. The ‘centre’ zone was set between beams 4-13, with the total area being 204 cm². The area not enclosed within the ‘centre’ zone was designated the ‘outside’ zone.

All animals were habituated to the testing room, with the equipment switched on, for at least 30 minutes prior to commencing testing. Mice were placed in the centre of the chamber and allowed to explore for a single 30-min trial. Ambulatory distance, number of entries to the ‘centre’ and ‘outside’ zone, and duration in ‘centre’ and ‘outside’ zone was recorded. Each chamber was cleaned using 70% ethanol after each trial and all urine and scat removed.

The y-maze test for spatial reference memory was conducted on all mice as previously described (Kraeuter A K., Guest P. C., Sarnyai Z. The Y-Maze for Assessment of Spatial Working and Reference Memory in Mice. Guest P. (eds) Pre-Clinical Models. Methods in Molecular Biology. 2019; 1916. Humana Press, New York, NY. https://doi.org/10.1007/978-1-4939-8994-2_10). The y-maze arena consisted of a clear Perspex arena with 3 arms (40 cm long×9 cm wide) with visual cues placed on all 3 arms of the maze. The visual cues consisted of black and white symbols printed on A3-sized paper and were attached to the walls of each arm of the y-maze. Visual cues were randomly chosen for pre-treatment behavioural testing and kept constant for all animals, and a different set of visual cues were used for post-treatment behavioural testing. The arena sits directly below a digital camera (Point Gray, USA). Room lighting was set at 50% white light and room light intensity recordings were taken by placing the luxmeter in the centre of the arena at the start of the testing session and was kept between 90-100 lux.

The Active Place Avoidance (APA) task assessing longer-term spatial learning and memory was conducted on all mice as previously described (Willis E F, Bartlett P F, Vukovic J. Protocol for Short- and Longer-term Spatial Learning and Memory in Mice. Front Behav Neurosci. 2017; 11:197. https://doi:10.3389/fnbeh.2017.00197). The APA arena (Bio-Signal Group, NY, USA) consisted of a 77 cm diameter metal grid floor fenced by a 32 cm high Perspex clear circular boundary. The elevated arena sits directly below a digital video camera (Point Gray, USA) and in the middle of the room with visual cues placed on all 4 of the room walls. The visual cues consisted of black and white symbols printed on A3-sized paper. Visual cues were randomly chosen and kept constant for the 5-days of testing, and a different set of visual cues were used for post-2A treatment testing. Room lighting was set at 62-63% white light and light intensity recordings were taken by placing the luxmeter in the centre of the arena before the start of each session and kept between 60-70 lux.

The arena was set to rotate clockwise (1 rpm). The shock zone where a brief electric shock (500 ms, 0.5 mA, track-dependent) is delivered through the grid floor, is set at a 270° (pre-2A treatment) or 90° (post-2A treatment) angle and width of 60°. This shock zone remained constant for the duration of the test and did not rotate. Mice were to actively avoid this shock zone while the arena rotates clockwise. If the mouse did not leave the shock zone after the initial entrance, further shocks were delivered in 1.5-s intervals until the mouse left the zone.

The position of the mouse in the arena was tracked using an overhead camera linked to Tracker software (Bio-Signal Group). When the centre-point of the mouse entered the shock zone, as tracked by the overhead camera linked to the tracking software, it received an electric shock. During the trials, the researcher sat quietly behind a curtain, out of view of the arena. After the completion of a trial, each mouse was returned to the home cage and the metal grid, clear Perspex boundary and underlying floor were cleaned with 70% ethanol. All scat and urine were removed following the completion of the trial.

Peptide of SEQ ID NO. 2 was dissolved in dimethyl sulfoxide (DMSO), and subsequently diluted in Dulbecco's phosphate-buffered saline (DPBS; 10% DMSO final). Pre-treatment open field/activity monitor data was used as the pseudorandomisation parameter for assigning animals to treatment groups. At 12-14 weeks, male and female 5×FAD and WT littermates were intranasally administered either 10% DMSO in DPBS as vehicle treatment or SEQ ID NO. 2 in DPBS (1 mg/kg). Treatment commenced immediately after the completion of pre-treatment behavioural testing and was administered every other day for 1 month. Body weight was measured every second day prior to intranasal dosing.

Post-treatment behavioural testing as set out above for pre-treatment testing was performed over 10 days where administration of vehicle or SEQ ID NO. 2 was continued every second day.

After post-treatment behavioural testing was complete, mice were sacrificed by intraperitonteal injection of pentobarbitone (150 mg/kg) and immediately perfused with 50 mL of ice-cold PBS (0.1 M) containing heparin (19.5K units/L; cat #H3393, Sigma). Brain tissue was immediately extracted, and olfactory bulb and cerebellum tissue was removed. The brain tissue was separated into two hemispheres with the left hemisphere fixed in 4% paraformaldehyde overnight at room temperature. The right hemisphere was dissected into hippocampal and cortical regions and flash frozen in liquid nitrogen. Kidney and heart tissue was extracted. The left kidney was cut into four equal pieces and flash frozen in dry ice. The right kidney was cut along the long axis into two equal sections and either placed into 4% paraformaldehyde for fixation overnight or embedded into Tissue-Tek O.C.T compound (cat #4583, Sakura). The heart was cut along the short axis into three equal sections. The base was flash frozen in liquid nitrogen, the middle was placed into 4% paraformaldehyde for fixation overnight, and the apex was embedded into Tissue-Tek O.C.T compound.

Cortical and hippocampal tissue was homogenised in tissue homogenisation buffer (2 mM Tris [pH 7.4], 250 mM sucrose, 0.5 mM EDTA, 1% protein inhibitor). Homogenates were centrifuged at 15000 g for 15 mins. Protein concentration in the supernatant was determined using the DC protein assay (cat #5000111, Bio-Rad). Supernatants were stored at −80° C. until analysed.

Western blotting was used to quantify IL-6, IL-10, TNF-α, synaptotagmin and synaptophysin expression. Samples were diluted in 4× Laemmli sample buffer (cat #1610747, Bio-Rad) to reach a final loading amount of 30 μg and denatured at 95° C. for 10 minutes. Following transfer, nitrocellulose membranes were blocked with 5% skim milk (in 1×Tris-buffered saline containing 0.05% Tween-20). The membranes were incubated with IL-6 (1:500; cat #700480, Invitrogen), TNF-α (1:1000; cat #ab66579, Abcam), or synaptotagmin (1:1000; cat #ab13259, Abcam) primary antibody overnight on rocker at 4° C. Membranes were incubated with goat anti-rabbit secondary antibody conjugated with horseradish peroxidase (1:6000; cat #ab205718, Abcam; when probing for IL-6 and TNF-α) or horse anti-mouse secondary antibody conjugated with horseradish peroxidase (1:1500; cat #7076S, Cell Signalling Technology; when probing for synaptotagmin or β-actin control) for 1 hour at room temperature. β-actin (1:6500; cat #MA1-140, Invitrogen) was used as the loading control for all membranes. The HRP-labelled proteins were detected using chemiluminescence (Thermo Fisher Scientific, cat #2106) and membranes were imaged using ChemiDoc. Protein band density was analysed using ImageJ and band density was normalised to respective loading control within each lane.

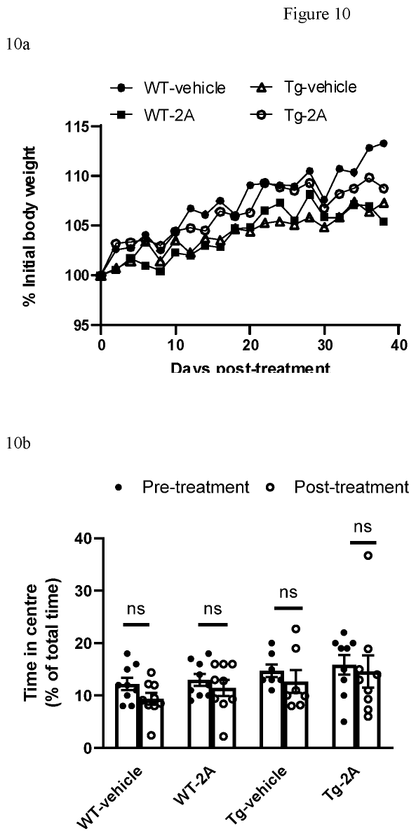

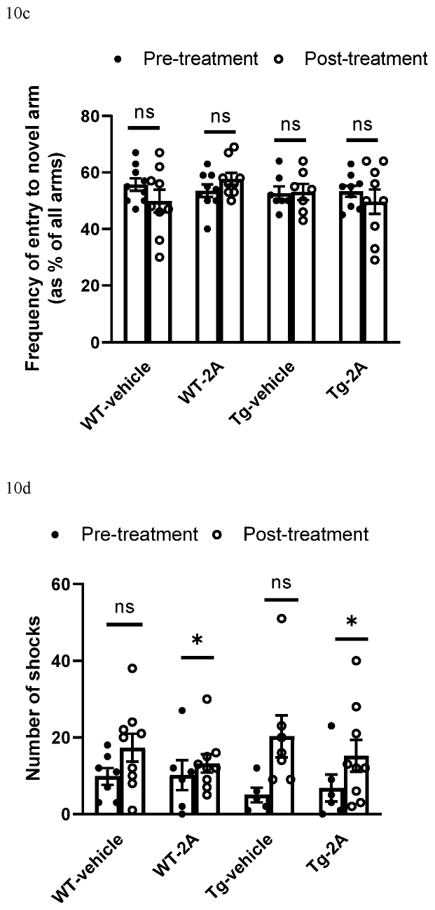

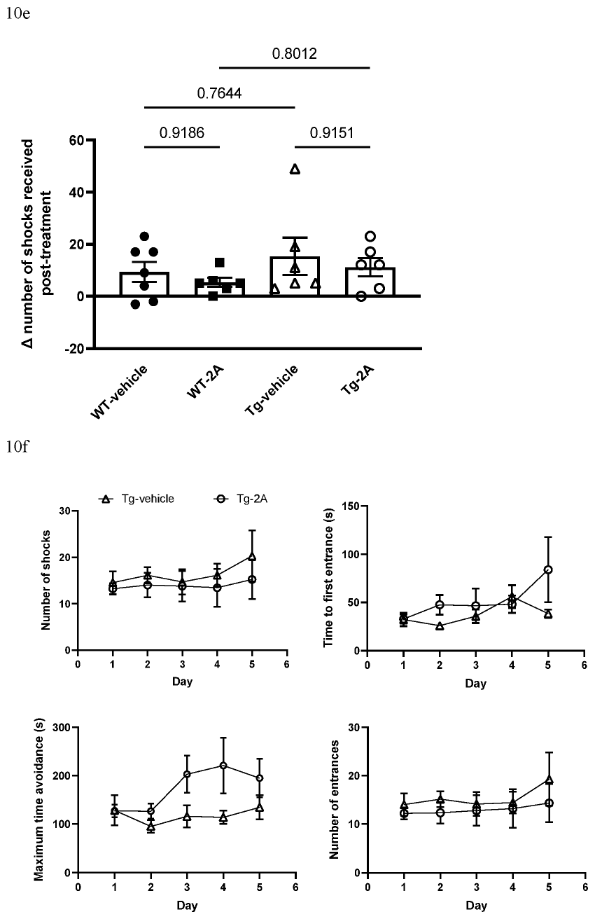

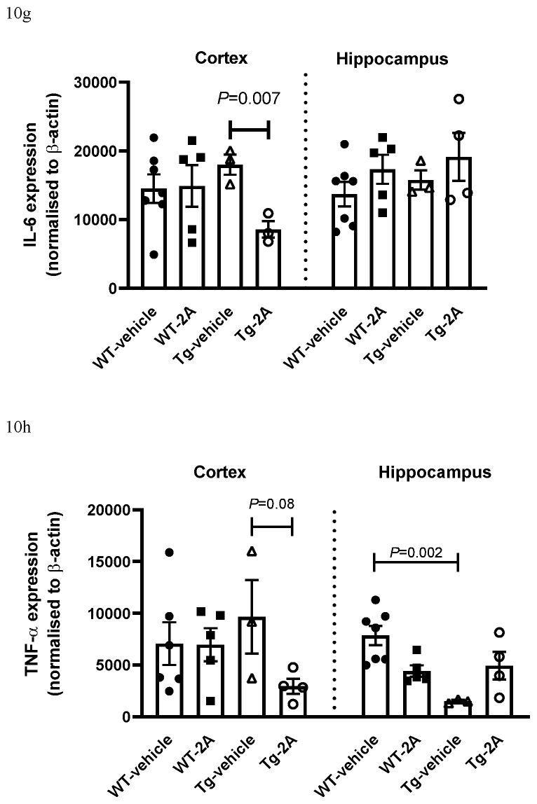

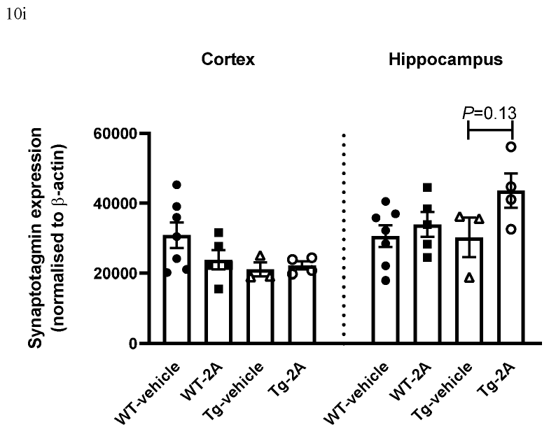

The results are shown in FIG. 10 (SEQ ID NO. 2 is referred to as 2A in FIG. 10). The body weight of the WT and Tg mice followed a similar trend whether treated with vehicle or SEQ ID NO. 2 (2A) (FIG. 10a). The time in the centre (Figure in the open field/activity test was not significantly different in each group of mice. The frequency of entry of mice to the novel arm in y-maze behavioural test for spatial reference memory, as % of entries to all other arms, pre-treatment compared with post-treatment did not show significant difference between groups (FIG. 10c). The number of shocks in the APA task at day 5 of testing was significantly more post-treatment in those mice, either WT or Tg, treated with SEQ ID NO. 2 (2A) (FIG. 10d). The number of shocks received post-treatment was not significantly different between groups (Figure Comparison of 5-day post-treatment performance of tg mice treated with vehicle or SEQ ID NO. 2 (2A) in active place avoidance behavioural testing (FIG. 10f), measured using (from left to right) (i) number of shocks received, (ii) time to first entry to shock zone (s), (iii) maximum time avoidance of shock zone (s), and (iv) number of entrances to shock zone. The Tg mice treated with SEQ ID NO. 2 (2A) had improved avoidance behavior. Protein levels of inflammatory markers Interleukin-6 (IL-6) (FIG. 10g) and tumour necrosis factor-α (TNF-α) (FIG. 10h) expression in cortex and hippocampal tissue of the mice were measured by western blotting and show a reduction in IL-6 and TNF-α in the cortex of of the Tg mice treated with SEQ ID NO. 2 (2A) compared to those Tg mice treated with vehicle. These data indicate that SEQ ID NO. 2 (2A) can reduce inflammation within the cortex. Protein levels of the synaptic marker synaptotagmin were measured in the cortex and hippocampal tissue of the WT and Tg mice by western blotting (FIG. 10i).

1. A peptide of formula (I):
R₁-Xaa₁-Xaa₂-Xaa₃-Xaa₄-Xaa₅-Xaa₆-Xaa₇-Xaa₈-Xaa₋₉-Xaa₁₀-Xaa₁₁-Xaa₁₂-Xaa₁₃-R₂ (I)
or a pharmaceutically acceptable salt thereof,
wherein:
R₁ is —NH₂;
R₂ is —COR₃, wherein R₃ is selected from —OR₄ and —NHR₄, wherein R₄ is selected from H and C₁₋₃alkyl;
Xaa₁ is absent or is a polar uncharged α-amino acid;
Xaa₂ is leucine or alanine;
Xaa₃ is phenylalanine;
Xaa₄ is glutamic acid;
Xaa₅ is a hydrophobic α-amino acid or a hydrophobic β-amino acid;
Xaa₆ is any α-amino acid;
Xaa₇ is a lysine;
Xaa₈ is a hydrophobic α-amino acid;
Xaa₉ is a hydrophobic α-amino acid;
Xaa₁₀ is leucine;
Xaa₁₁ is absent or is a hydrophilic α-amino acid;
Xaa₁₂ is absent or is a negatively charged α-amino acid; and
Xaa₁₃ is absent or is selected from serine, threonine and cysteine.

2. The peptide according to claim 1 wherein one or more of the following applies:
i) R₂ is —COOH or —CONH₂;
ii) Xaa₂ is leucine;
iii) Xaa₅ is selected from alanine, isoleucine, leucine, valine, aminoisobutyric acid, β-homoalanine, β-homoleucine, β-homoisoleucine and β-leucine;
iv) Xaa₆ is a hydrophobic α-amino acid;
v) Xaa₈ is selected from isoleucine, leucine, methionine and valine;
vi) Xaa₉ is selected from alanine, isoleucine, leucine and valine;
vii) Xaa₁₁ is a polar uncharged α-amino acid;
viii) Xaa₁₂ is glutamic acid or aspartic acid; and
ix) Xaa₁₃ is threonine.

3. The peptide according to claim 2 wherein Xaa₆ is glycine, alanine, valine, leucine or isoleucine.

4. The peptide according to claim 2 wherein Xaa₆ is a hydrophilic α-amino acid.

5. The peptide according to claim 2 wherein Xaa₁₁ is selected from serine, threonine, asparagine and glutamine.

6. The peptide according to claim 1 selected from one of the following:
SEQ ID NO: 1
H₂N-SLFELGKMIL-OH
SEQ ID NO: 2
H₂N-SLFELGKMIL-NH₂
SEQ ID NO: 3
H₂N-SLFELGKMILQ-OH
SEQ ID NO: 4
H₂N-SLFELGKMILQ-NH₂
SEQ ID NO: 5
H₂N-SLFELGKMILQE-OH
SEQ ID NO: 6
H₂N-SLFELGKMILQE-NH₂
SEQ ID NO: 7
H₂N-SLFELGKMILQET-OH
SEQ ID NO: 8
H₂N-SLFELGKMILQET-NH₂.

7. (canceled)

8. A method for treating or preventing Alzheimer's disease comprising administering to a patient in need thereof the peptide according to claim 1.

9. The method according to claim 8 further comprising administering an additional active agent useful in the treatment or prevention of Alzheimer's disease in combination with the peptide of formula (I).

10. The method according to claim 9 wherein the additional active agent is L-arginine, L-glutamine or an antioxidant.

11-12. (canceled)

13. A method for treating or preventing inflammation, fibrosis, lung disease, hypertension, pulmonary hypertension, cardiovascular disease and/or renovascular disease comprising administering to a patient in need thereof the peptide according to claim 1 or a pharmaceutically acceptable salt thereof.

14-21. (canceled)

22. The method according to claim 13 wherein cardiovascular disease and/or renovascular disease is diabetes related.
